SEQUENCE LISTING (6 sequences)

SEQ ID NO: 1 - DNA, 14770 bp, Legionella pheumophila
gatcaccccgccgggcgatgaaaacgacattgccatcaatttgggctctgtcattagggcaacagacagcgacggggatacggtagtggctgcggccgatgggcttgtgattacggtggacgacgacacgccggttgcgagtgcaacgaaactgactggcgtggtggatgaagacggcctggcaggcggcattgccggtggcacaggcgatgtggccggtcaggcggtagcggcttcaggcaacgtggctgccttgttccaaagtggcgcggatgccccgttaagctattctttgaattccagcaccagcggcttgccggctttaagttccggcggtgtggccctgacttatgcggtgtccggcaacacgctgacggcgtctgcaggcagcacgcaagtctttaccttcactctcaatgccaacggcaattacacgttcaccttgctggcgaagctcgatcaccctgccggggcggatgaaaacgacattgccatcaatttgggctctgtcattagggcaacagacagcgacggggatacggtagtggctgcggccgatgggcttgtgattacagtagacgacgacacgccggttgctggtgccataagcgtagtagctgatgaggacaatttaccgagaggtaataatgacgcagcttccggagatgctgctcaaagcaatttaactggtacattgcctgttaattttggtgctgatggtgcaggtagcattgattttcaaggtatgcatggtctgagtactgtcataggcaatgacaatattacttataactggaatgctggtacgaatacactgacagcctatcgaacaggaggaaccttaggggttaacgatgtgtttaaagtagtagttaatccgacaacaggtcagtatacgtttactctgctggctgctattaatcatcatgctgttgctgataataccgaaggattggttgatccatttgtaaatcttaattatcgagttattgatggcgatggggatactgcaattggaactctaaaagtaacgattgatgatgatatacctaaagcatttacaccggaagaaggttttgtaacgaatcaagctggtattgtaagaacatttgatctggattttgatgccaatattgataataacgtcggtgctgatcaactcggtacaattgctttttcaggtatcactaacgggcaacttgttacagggacagttgatggtgttcctaatcagactttaacctctggagggtctgccattcactattacgtgagtggtaataatgtggttgagggatggattaatggtgggcctggtgacgtgggttccacaattgtgtttaggacttactttgcagccggatatgaactataacgcaagtaatgacacctataaatttgaactgttccagcctattagtacatcaacgaatgtgagcattacaaattttaatggggttaatgcaactagtagagagttcatatatcttgagaatgctagtgcccccggagaagatatattattttctggttacatacgaaacgataatggcagctttactgatgctactgttaatacaagtactcaaggtataggcgtcaataatcaaaatatgaatgatcgcgagaatttgcgccttgattttgtcaggaatgcctctactaatggcacaacctccaatatgacttatgaatacgatgatcattataccgttaataactttagttttaaaattgttcaggttacaggaaatcctccggcaggatcaatagaagtatgggttcgtgcttataatgcggatgatgatgatccaactaacaatacagttagttctgcaaataatttagcgcatcaagatgcattgagagatgatccgcaagtagcgctcacacaaattttagtgaatggtgtgcccggttactccaactgcggttaatgctagtggtgggtatttaattagcggcttaaatttaaatgatacgataacacttagatcagctaatggatatgatcgcgtagagatagaaaacgcacgttcgggagctcatggagtttcaaactccagtttaaatggtgaaacatttgatattggtttattctcatacaatacaattaaaacgactccttctgaaatcaatattaatatgggtctctcattgacagactcagatggtgataaaataacttcttcaatagaaattaatctggctccttcagtatttaaggtcggagaaaatgtcgatgatacttccagctctaatgttcttcaccgcgtaggaggtgacactggtgttgtcgatggttctggtggcgcggatattttggttggtgatgttggcggtgtagagattgttggtactactgctcgaattgcttttgtattggatgaatcaggtagtatgggtcaaaatttcggaggaacaactcgtttagaagtactaaaacaaacgatgactgatatattaactgagttaagtaatacgcctaatgccagcattacagttcatttagtcaaatttgcatcagtagtgaatggtacaggcacttttgaaattactggtggagaattgcaacaggctttagactttatatctggattacagatacaacaaggtttattagctggaacaaactatgaggctgctttagggcaaaccttacaatggtataatagtcagtctgggacagcggatgtgcaacaaactctattttttactgatggcgcccctactttttatatggatgggaatagtactgaatatacgaatattgctcgtgtctatggaaatggaagccaaacagaggaagttctctgggaaaatctattcggtgagcactctggaggccaagccacatcagatcgcattaataatctcacagagtctgaccccagagatttggatggtcttcaaagctattctgttgacactaatggtgatggtatttttgaaactcaatcggtaaattcacgtagttctggaacaacacaaactacaaatgactacgtaagaagtattgcagatactttcaatgaagtagacgctttgcaagcgtatggtcctttgcgtgcggtttccatagcggataattttagcgtttatttacaagaaatagatagtacaggccaaccttatattgctgactctcctgaagtgttacaagatatactggatgaattaaatccatttaatgttttgcttgcagcgggtagtgataccattcaagctaatgaggaaaatgatttaatctttggggatgtgttattcactgataaactggctgaagatgaaggtcttgatttacctaaaggatcaggttgggcagtatttgaagagctggaagccaatcatggatggagtagacaagataccttggattatataaaaaatcatgctgatgaacttgggagagaaacagtactctctagtggtaataaacgttctggtggacatgacaccattggtggtggccagggaaatgatcaaatttatggccaggaaggaaatgatgtcatagatggtggttctggaaacgatgtcattgtgggtggaacaggtaatgatacgctcaccggtggctcaggtgctgatcaatttgtgcttgtgaaagggcaaggaggtgcgagtacaggtgtggccccgatcgacactattacagactttgaagtgaatattgataagattgttattaacggtaataacattactggtgtcagtgtatccactccggtttctaacacttatactataacggttaattattctgatgctgctacggaacactttaaagtgacattatccaatggtgctttattgaatgactctggaaaaactcatttgaacggagtggttataagcggtggcacagctactattgatggcacaatcgttggcgccgttttgtatctggatctgaatgccaataatcaggaagatgaaggggaacgactgggtattaccaatcaatatggtcatgtggaatgggtcgttgatctctctaaatttgatgtcaacggtgatggtcagtatgttataggcgaggctcgtgctgttcaaacgggtggttttgatatagatacgggtttaagttatgaaattaatctttacgggccagtaggttcagcagtcatatcaccactgacaagcctcttgcaagctcagttagaagccggtatggattatgagacagctaatgctgctttggtcgcgcgcttgggactgcctgagggaacacaaattatcagtttcaatcccattactggcagcggtgaagtcttggctcaaaacgcaggtgtaatgactgcggccattcagtttgctgaaatagccgcaatacactattcaaccgatgaaagccatgtttcattcgctgtctttgaggcaattagcaaggcattgcttgaacttcctgaaggggtagtcgctgattttggcgatgaaggtttcttgcagtccatttcaaattatctgggattagatgctctggttacctctgatatgattgactatatggcagctggccagaaagcgttagaggccagtattttgactcttgcgcccggagaagatgctctgactgccatcagcaagatacagcatgtgacccaaggtatatatgcccaagctattgaaatggcattgatagggtatttgccattagatgctcttaaaaacatgagtgctgttcttaaggcttatgctgatggagatattacggatgaacaactcaattcttttgaagacaagctttccgctgtggttgtgaattgggaagataacaatgttacagacacatctcatcaggcttcattgcaaacagatcgtactgttgatgacgcttcagtcaaatcagaggtgaatcatgaaatggcgcaagaagttaatgccgtgattaatgattttatggctgaacataatatttctttgtactattatgctcaacaggctcaaatgcctcaagaagcacaagaacaacaaccaacagaggatcaggctgaagagacggtcatgcatgacgacgttgttcaagatttgttgcttgatgatcatagccttaccatgttagctaataatccccaattaaacggtggtaatggcaatgatgttctgcatggaactaccggaaacgattttataagaggcggtcaaggaaatgatacgatgacaggtggtggcggtgttgataccttcttctggttaagtggcgacgatgatggtggtgtggataccattaccgatttcaaagcaaatcctgttgataaatcatcagatgccagcgtccttaacttaagtgatttgctatctgatgcggatctggaaaccaatagtttggataattatctgaatgtttctaccacagaagagggtgatacagccattaaagtagaccctaatggtaatggaaactttgacgctccagcgcaaacaattatattagaagacgtggatttaacagctgtttttgctaccaacaattcgcatgatattgtgaatcaaatgatcgctaatggaaacttggtagtagaacagtaagatgaactcattacattggtgaatatggaattcaccaatgttttattaattggaaaatatcctcgcgacatttttctcgctgctatatactcatgtgttcattcgataataagccttgcaattcatttcacgttattaaattgatgaaaaaaggcaaaaaacgctatggagcagtatcatgtctaatccttatatcttggttttatactattctcgcactggctcaacggcacaattagctcaatatattgctcgtggggtggaaagtgtgtctgggcttgaagccaggttacgcactgtacctcctgtatctaccacttgcgaagcggtggataagccagttcccgatgctggcgcaatttatgtttccttggatgatttgcgtcattgtcatgggttggctttaggtagccctacccgctttggtaatatggctgctccaatgaagtatttttgggatggaactacttctttgtggctgggtggagatctggtgacaaaccggcttgtgtgttttcttcttcagcgagtatgcatggtggacaggagactaccttaatctccatgatgattcctttgttacatcatggcatgattttattaggtgttccttatactgaacctgccctaaccagtacgcagtctggtggtacgccttatggtgtaacccatgtggctggtggtgataatgagaatccattaagccaagatgagatttcgttggctaaagctctgggggaaagattggggcgtgtagcaatgggactgaagttttgagatggtttggatgtttatttcccctctccccacccctctcccgcgaaagaaattgtataaaaaaatttgggtatgattcgcgggagaggggtggggagaggggaataagtggttccagctgatgctaaattattgaaacaagcaaaatctctccgggaaaaggagcactgatgctgaaaattgtttatggtactatctaagagcacatcgattaaaaggatacaaatttaaaagacaagttcctattgggaattatattgtcgattttgtttgtattcagagaaaactcattgttgaactggatggtggacaacatcttttaaataaagagtatgataaagaccggagcgcctattttagcacattaagttataaggttttgcggttttggaatgatgacgtgttattgagaactggggacgtacttgagagtattgtcagatgcttggaagaatcataaggttgataagattaacatttaactgttcagtaatgacagcggagtaccctaagtaatgaaagtaattacgttttaacgccaatggcatacgctcttctgcccgtaatggattttatgattggcttgtagggcaggatgttgattttgtatgtattcaggaaactaaatcgcaagtagagcaattaatcccggaagaactttattttccaagggattatttttgtgattattttgatgctgaaaagaaaggctacagtggggttgccatttattcaaaatataaacccgtgcgtattgtgaaggggttggggtttgattattgtgataaagaagggcgttatatccaatttgattaccctaaattaagtgtgatttctctttatttgccctctggaacaagtggtgaggatcggcaagaagtgaaatatgcttttctggagcaatttgccagacacttgatgaatttaaaaaatgagggaagagagttaattatttgtggtgattacaacatcgcgcataaaaagatagatttaaaaaattggagagggaaccagaaaaactcaggatttttacccgaagaacgtgcctggatggatgagttatttggttctatggggtttgtagacgcttttagggtacataatcaggaagaagagcaatatacctggtggtcttatcgagctagagcctgggaaaagaatgtcgggtggcggattgattatcaggtgattacgcctggtctgactgaatatgtcgcggacagccgaatttttcgcgaagcacgtttttcggatctttttgcatgtagtaacaaagcaaacttggagacacgccaagttcattagcctctttactcgtaaaaacaggcttttccagaagagattcaatgttagcaagcttagtatgtgttttcatgtttatattttaacaaataaacccaattaaatccaatgggttcattttttaaattttaacaaataaacccaataattatttttgggtgtatttattaaaataaaattattatgtcaaccccaaaagccatcttgaatcagctcctggtttttaataagaacctaacaattgtaaacttagtttacaaaaactataattaattgtgttgtatgctctaggcaactttgatcatgagtatttacaaagtagaaatatgatgaaaactaaaaccgaatcatcgtttttgtataaaggcttaggatttcccatcgaattggagcatgtcgcaatgatctgtatcaacggagaatggatgcccaaaattgacgttcgtaaaattgccgatgaagccattaaaaaacttgccattcaagaagagagattaactggcaaccaagttaaattcattcgctcctatttttccatgacccttcgagagtttggaaaaacagtggttcatgaatcgcacaccgctgtcaataaatgggaaaaatgcggtaatgatattaccaacatgaatgaaaataccgagcaagtgttacgaatttacattattgaacagacacagactaagacccaagccgaacaagagaatttctattcaaattttaaaaaaagcaaagtatttgtgaatgctgaacgcaaaaatcccaagccaattcgattaaatctctgtgcataacggctcacccctgctctaatcaaaatcagacaaagtcttaccaaatttaaataaatggtaagactttgtctgattttgattaaattaaaccgtgacaatttgtcatagtttgaaaataacgtcatatatgttcaatcaataacatgtagaaaaagcctttgacaaacacccagatttaaataaacatcatcaatagataaccattaaactaaactttcaccgcaatttttttaactcccaaaatcaatgagcctaaatccgtccaaaacaaggccgaccttaacccgacacatcaacccgacacaaacccgacatagcaagattcagtactgacaagaagaaaaaacctgtcgaatagaaaaacccgacaaagagaaattctctaggccgacccacctcataactccaaaatctgttttttatgtttttacgcgcgcgaggaaaaattttttaaacaattttgaacaaaaataaccaattaccaatgcaaaaacgtctcagtaaaaaaatataaggttcattgaatatcatttagtatcaagctcgttttccctattgacactcttcaccaatttcctagaatgaatcaagtttaatgcatcttggttatctaaacgtcatcgattgatagacgtttgatatcaattgaaataaaaatacagggttaaaacaggagtaaaaatggatttcttattcgtcaccacatacgaactggacgcactcagcgaaatccccctcatgcaaagagtagtctatctcatgggtatccgcccctacatggacagaaagacattcatagtcggcattaaacgaaaaatcagttaccagtcactgagagaaacactttatgttgcaccaatcaaaggcgtaaaaacgggctgtcctagtcaccaacaaatgaaacgcgtaattaaatctctggcacgccaaggcatcatagaaattcgctcaacagcccaaaatctatttgtgaaatgtcttctggcaaacccatatgagccgatagagattgaaaatgaatctcagcctaaaccaaatccatcaaagcaagcagaacagaatcaaccattaccatcagaaaatctgaaatcaatcaaacccgaaaaatcatcctactcaaaatcaggcttaagcgcagccagcagtaacctaaagagctataccaaaaattccgaacaattctggcaactctaccccctccctagctataaaaatgaagcatggaagcaatttcaacaaattgatcctgacgatcgtcttttttcacagatcatggatggattacaaaagcagttaaccaaaattaatcggctaggagcttattggccttatcctgctagttggttgggtcaaaaaagttggtttgagaattcacataagtagttaaagccgcctaagtctatatgcactccacaaaatcaattcaatttttccagaataaatgttgttcccattcagctggaaatcccatatcttgaatacttacataattggaatttggaaaggagtgtatcagctccactagacgcatttgccatgttgatcgcgggcaaattattttcatgtaatgcgccaatatacataaaaccacatatattctcgatgtcatatgaacattatcaatgaatgaatcgaaagcaagcattgcacctggctttgcaagcttcggctgatcaattaagtttctattccataaacggtgtggtgcgccgctacatttcggacataatttaaagacgtaaccaactctccataatctgccagtcaggaatgtcatatttgttagcaatgatagccttatctttaactgccattcctttataaaatactgacaaaagaccaaagtcccataactcaatagcaacccatatcggcaatggtaaaccgtactttaccttgtagtgctctacaaaatcttcttttgagcgatttattaattgctcatgtttagaaatccattcttgatacttcgttttacccgtatttggatcaatttttttagtaaaatgaccatgtaataaatcaggattggtgtaggcaaacgggtctctttcaccaagcaaataagctatgtccactctgaaagcaacttctattcctcaattgcatccaatattaataggcgcagctttttatcaaagacatacaaatccacaacatcctgaaatcgagaacctggcaaaaactcatctaaacgccgaggtcttacagattttgtttgttgtactactaaagggagtaatttacggcagggataccaataaccacttaaacgatagtagcccaatcgacctagatactccaatgccactttgtcattggttacctccaaaccacgagattttaataactcaagttgctgttcaaaggtaagataaggtttattataatgcgacattttttaggctttataaaaaagaaaacccgctcgtgcatttttcaatgtagaggagcaggccgtgttatctatagtataacactatttataatgttattggcaacaaattgaatagttatcattacatttttaaaactacgatgagttatagcaagataatggcttaaagccaaacgcaagatatagcccgtaactctttaattatgtcttatctgctgaaataattttcttaatcacttgaatgctatctgataaattcttggataaagttctgtaaccctctgttatttgctcttggcttttattagctaaaaaaaataactgttcactagcttgcaataaatttataactaaatcagattttgaagagatttgattataattagcccatctttcaattgccacatgagataaactaaataatggttcacagggccaagttttactgtttactatatttaatatttctctttgagtattaaccctattttttaattcactcatataccacctctctgatgtttgatatcaaacagttcatccagcgatttatcatctctaaggtaagcaatagctcattaacttcctcagaagacaaggcatcccccaatcccctggtagtcaatattcttgccttttcaaagagattataattagatctagaaattggtatattcatctcattaagattaaaagcagaatttcttgaaaaatttggtttttcataaagccctagagtataacaagacgcaatattacggattgctaaaaaaatcattgataaatcatagataatacaattactttcaccagttaacgatttatatgcgtcctggaaaattccgtaaaagttaagacaatctctcaaacaatttttatattgtgctggcttatttaaatcctttagaaaatcacaaccatcagagctaaaaattaattttgactcccaaaataaatgccaagcaaaaggatttccactcatccaaagatctgtaattttattttgcgtataaattgaatataataacttattgaattgattgggtttatttgtaatagctaataaatcaatatcagagtatatgtctatctctcctctacaggtagatccaaaaacataaatgtgcataaatagttatctcctatttaagcgttttataattaatgataaaaacattccaaagataagtaacctaataatagtaatgacagtaataaataaattggaatatccttgaggatgatacaccccaaaaaaaatggagggtgcacttgatagcgaatcacgaatattacaatttggggtcgctttactaatatcgattaatgctataatgagaatgacaaaaatcaataatctaataattttccaggccttttcaccatttccccaaacaaaatccaaaaacttaaaattaagccaatcaagaaaagcagcaaatcgttcaattccttgatatttttccctataataatattctttagaatgccatgatttttttaggtgggtctcggtagcttctagttcaattttaatggctaaatttacagattttgaatccccaatttgttgatagtttgttctaagagttcgagcaaagtctctctttaaattttcttctcttggtgctgatttatataaaatatcattatcaacaagagttttttcaaaaatgcaatattgaaaattacaaccaggaaaactagaaccattcaaatttgaatttataaatttgcaacctgtaaagtcacaagagtcaaatttgcactttctaaaataaacattatcaaaaatacagtatttaaaactaactttagtgaaagttctttcttttgctgacacttttttgaaagtacgtttgtcaagaatttcacctgaaatatcgttagcaataaatttatcttcaatatatatccaactattttcttgaaacaatgatgtcgttgttgtcaatttcggatccttccgttattaatcagatgaaatctaaattagattatgtcttaaaaaaattaaaatgaaaaatgataagctatgcgtcactgcaattattctgcagtgacgcaaacaaacttgctataaatctaattttaaaatcaaacaattaacaaacaaaattcaatcccacaccacaggattgaacaaaatattcatccaaaacctattgctcccagatctcacctttgctaaatttttcataagcataactgatgagcctgcgcaactcaggcgaaaccctagggtcttatgctaagccactctcattcccctaacctgtggctggacgtctagttgggaatctgtagttctaggataggctaccatcgggtagataagttaataatcacttctttattaaccatggatcagcggcagctgctgttggattgccgtgcgtttttgtatttaaacaataaggagataaaccactaaaaaaccgttgtaccaaaataatcattaaccttgatttgccccaccatttcgagccttgcggcacttgctggatatcctctttgagtgaatcgctcgattaacactaaaccaaagacagggcaaataaattgattgaatagaactatgcgcaaacactctttaagacaaatagcgagccagtatctggatcatgataatagcggtagccctcgtggcaaaaaatatcgtcgctttgttattcttaggatgattgaggatctctttgttcttggccttgcgccatccaattggccaggtttgacttccctccatttgcagcaacttattcaacattggcacaagaaaaaggttaagccttcgactcttatgaattacatgaccatcatccgcaaatttctatgtcacgtgggtcataatgctgagaatattgataatctcagccttggtttacaaactaagaaaattaagaagaaaactcggaaatcccctgctgatatagtagagaagatcaatgatcctgtggccaaagtccttttaggctttcaaattcattttggtttgaccttgagcgaagccatgcgcattttgcccggaatacatattcaagaacatgaattattactgacccgagaaatcacttttaatagcaaggataggaagattcctattcgctctgaagctcaaataaagcttatagaggattttaatctattaacccaagggaacggttgtcttatcagcacccacggatatagagctatctgttttagctggcaaaaggctatgaaatccttaaggctctctggaaaaaaatgctggcgttatctctatgcccaacaattaaattcacaattatcctctcaaatatcccattaccgtttgtccttactcatcatggacgaaatgggattgaaatcacgtaccacactctggagttatctcaatgagcaataaactacgcagtgcactttattcagtacatcaattaattaaacaaatgcagggttattcctatgccagtaaggcagatatgaaacatatgattaatcgctgcatgaaagatctacacgaattgggttttaaagtaggtcatttaaatggcctaaagcctaaacatatctatgtacttgttgaacattggaagaagcaaggcaagagtccagcaactataaaaaattatatgtccaaattgcgtaaactcgcttattttctggataaacctaagctggttaaaacgggtaatgactcttatcaaattgataaacgcacttatattcccactcaaaacaaagccatccaccatatcgacttaaacttatgtactgatccacatattcgcctatcacttgaagcacaaatgctttttggcctacgtagggaagaatccatgaaatttgtagtcagtgaagcttggcatggtgattgtttatatatcaaacccagttggacaaaaggtggtattggtcgattgcttaaaatcaccaatgaagagcaaatgaaatggttatcaaaagtctggcaccagataagacg

SEQ ID NO: 2 - DNA, 6920 bp, Legionella pneumophila
gatcgagaaatctgagctcttcatgccgcaacattccatgattgccaatcaaataacatccagccccatttttattttcaattgatctaaccaagaactattacgattggttgtcggtaatccataaaatccccaattatcaacaacctccagcaatttactggtttcgtccatttttgaaagcaatagacaactgtgccacagtggattactccctacctcctgatctgtagtacaaaatgtaacagcatatttcatatcaccaccctcatcgatttgcatctaattatttgaccttgattaaatagaccttttgcctaaagccgattctttcattttattgctgctttgctaacattcctgcattgctcattaactgcattataaactccgctcctcaaaacgtttgcaccttgccctaaaaaataattggcttaggcataaagcctaactaacaatttaaatctctaaaagattatttttaaaattacaataaggcatggcttcactttcgtaaatagccaagatgagcgcaacccttcttgtgtttaatataatcgaggcaacaaatgcaggctgggccttgtggcccagaaaaatcggataattgattagcaaaattgaaacatcatctgttaaatagctcaaagagccgagcaatgatcttaacccaagttattcattaatttggcttgattcaactgctcataagctcgctttgcagggataaaattgggatattgcgtcaccagacgttgcaagatcattctggacaaaacctcctccccatgattccattcatttaatgcatcaaaataaatcatatcagcgctttggccatcaaatttgggaacattattaaataacacgggctcgacataacttactttaccctgggtttgtgcatctaatctttgcaatactctacgggcctgtaaacttccgttttcactcgcttgttgtaatagctcacggcctttttcgggtagtcgttcgcctgccgttccctcaatataataagtacctaattgatattgagcataagcattttgcttttgtgccaatttttgataaatactcgctgatcttttatgatccttttcgacaccaagaccataatgatacattcttgccaaagccagcattgctttttcattacctttatcagatgcatcctggtaataccgtaaagcatctggaaaatccaatttaacaccaacaccagtttcggataataaaccgagggcatacaaagcattcccattacctaagcttgcagctttcttataccagaccaaagcttgttgttcgtttcttggttgccccaaaccataaaaatacattccagccaattgactcatcgcttcaggagcacctttctctgatgcttctttaaacaaagataacgctttggagtaattgacgggcacgcccttgccatactcatacattaaagccaaattataagcgcctagcatatctccttttgctgcagcattttcataggcttttagtgcttgagcataattatcatctaccgtttcataaataaaacctaatgctacatcagctttagataaatgtttcgctgctttttgataccagtgtttggccaaattataatcaggttctcctgtttcacctagttgataaaattggcctaataagtattgtgcctcaggattgccttgttctgccgaagcagtataccaatgttgcgcgccagcaagatccggtgcagcacccaatcctttttgtagcatataagccaattttaattgtgcatattgatcgcctttatcagctaagcccttataaatttccttagcttgattcatttgttcaggaacactgctttgagataaataataatctgccaatactatccccgcatgactattccctaatttataagactcaattaaattgggtaaaaagtcttccccggattgttgtttaagtactgccagattaaagtcggcataggaaaattgggagtctgctgattgcttcagcaaatccaatcctttttccttatcctgtgccaccccttttccttctgtgatgtaagtgcccaatataaactgacttacgggattttgtccggcttgttgataccagtacatggctttggctggatcagcggttattcctataccacggtcatagagcattccgagtaaaagagcggcattctcattgcctgtttcagcttgctcttcggctactttaaatgcttgttcctgcttttgcctgttatcatccatcgcattataaaatgccaatggcaacaatgcctctgccactttattcttcacagcaccttggtataactttcttattaaggcaattttttgttttctgacatctacacttaaaccattgttgtagtctcttgccaaatattctgccaactcatattctgccggcccataatcattggcagctgaaagataaagcatggccatagcttgctcatcatttgctttaatatactcttttccatcaggaccaacgatcccctgacgcaatatccttgccaatacgtattgtgctctcttattccctttaaatgcggaatcagtcagccaatttaatgcttgttggtaatcattttcgtctttaccgcgtttcagataaagtattcctaaattatattcagcacccaagtgcttttgttctgcggcattctcataaaaaataatagccgaagcatcatcttgcattaaaccaatcccatattgaaacaattgaccaatttcaaattgagtttgagcatttccgagtattgccctgaaatacaaataattgaccacagacatataattgacagtccgtttccaatcccctgtccaaatgtccatcaaatcaaaactgccaacttcgctatcatcatcataattcgcgtcagcataaggaactggcaagttgagcttttgtacgacaggactattggctctctctagagcttctacaaatgggttaagtctgtaagttggataagcccattgatttgattgaaactccacttgtttactgattaaagcatcatagtaattgcttataggaatactattaggctgagtcaactcaaattgtggtttgaaaatatcctgacgagagatggtttcaagcttaggtgcttgattatagctattatttctcagggcagacggattattccatgggctaaatataccattcatctggtaagccgtctcttccaatttatccgttctgtcattacttagccataatgcagcttgggcaagaacaggaggctctgcatttttcttctcgttttctgctgctttaagagtccattgtttagacaattcttcatcagccgctacgccaatccctttttgatataaattggctaattctttttgggcttgaacagaaccgttctgagctgcttttagtaccaataaaaaacctgtttttggatcataaactggacttttatcttgtaaataaatatgggctaactcaagagtagcttctgtactctgttcctcggccttattaagccattgaatagcctcatcgtattgattttgcattaaagccaatttacccaactcaagcatggctggagtatattgttctgaggctgctttatttaataattcaacgcccttggcagtatccttggcaaccatttgtccggcgacataaaatctgcccaattctgttaaagcttttggatctccattttctgctgctttattcagccaaatgatccctaattttttattacccgcatgcttactctctaagaaatactctgctaaggtgtattgagcaataggattaccattttttgctgcatcaatataataacgtcttgcgatatctgtatttttcttgacgccaacaccatataaataagctgccgccatatacatttgtgcgctcacatctcccaccgcagctgcttgcttaaaccaacgagcagcttcttccggatttttttcatggagcaaactgtatttggccatgagcaagacagcaggtaaatagcctttttccgctgatttggtaaagtagcgcatcgcaacacgattgttttttaactgcccatatccatagagataaagtcgccctaaatagtattcggcaacagcatcctgacctgatttaatgattaggggctcagctgcttcattataattgcctaatctataagcattgaacgcatcattggcaaaagcctgttggccagctgacgcgactaaaacacacacccaaggtactagtgatttcatgaagattcccctcttttcatggccatttatacaccgggtataatgccataatgcaccacttgactaactgccccatttctcgtattgctgtctactatccaaaaattacctttattattcaaaccaaatttgacattaacatattcgcatactttaatcacatcagcacaatccagtatttttccataggactttgccatcacccaaggtacagatgaacttcacttccttttcacctgtggccaaaacagcccaggaattccctctgatagagtgatttagccaataggaatataaactttccttatcatgcatctgatataagctaacggtttctggcaatgtattataaataaaatacattgactgcaaggctccttccttccctggaagaagagtcaatacctttaaattttgctgatagccaacagcagtacaaccgagaggaaattttccgttatcttttttctccgccttatcttcggcggttgctgttttattagatttggcggcatatgccgacatatttagtgcaccagttaatagaattacaatgattgcagttatcgagcttcggctttttaggttcattttttattctcactatcatttaaaggacgtacaatgaccttagtccccataaaattgttacgctcactggacaactgctacaaaattttcggtttaaccacttggcatcctggataaacatacgcacacagaccatggctagcccttacccccggaatatcatcagaaccatgtaaagcaaaccctttatgaaaatacatgcaataaggcattttgcaccgcctctgcctataggaaaaacatctgaagtacaacgaacattttccttgctcgaatacacggaaaattcctgtcaaagtacgacagatctttattggaatcagagcatttatcacttccagaagaaatgggtccccaccatactaaattgccctgagcatcataggcgccccaagccagcttctcttgatccacgataatttgtttttcattttcactctgaattttaagtgggaaaggagagacatcaaaaatattgatgttggctaaattctttggtacagcgatttctctgcctgaccaaaggtagttataagtacgattgactctttgcacaatgtctctttggacaggatcagggaataatttttcccaactttgtcccccggttaccttaatacattcatattgaggataagcacataatgccgttccataaaaatgcccagcgttaagtacagtcaccattgacatcatgaacgttgttaatagtatttttttcatggtgccgcccccttatgcctgatttttataattttatgcaaaatttgtgctaaaaatgaaatgccactctttttatcttttttcggttatttttaatttaagtttagacccatacaaaaaaaaattgaaattattgctatatttaaattagcattgactgggttgaacactcatgagtcctttaaaaagtaaaattaaaacaaagtctttcaacaaattaacaaaaactcaagacattaccaacacgattgaaagattatttgcaaactctcaagacagcatcattgtttttaatcaacaaggcaaaattattgctattaatgaaaaagccgctgctttatttaaaaaaagccctgacaaactaattgacaaaccgatttggaaactatttaagttaaatagcttcgctattaagaggcaatttattcaagcaaagaggtgttttcttttagccaatgaagggatagcccagcaattcacttggcttgaaagcaaagcacaaaaaccagtacttgcataccatatcctgttcaataaaacagaaatagaagacacatcaatcatttttaccaaacttaccgatattctaaaatcgaaaaccatagaatggattttatggtctttaactaaaataagtaaccaccaggaaatcaatgaaattattgatcaaattctacaattaaccagtgatgtttttgcagcggattatacttctgtatctttcataaacaatcaacaaattgcaaggacaatcagctactataaatttggaaagaaagaagagaacattagttttctttaatcaatacaccctgcgctaaagtaatccagaaaaaagctatctgttattttaatgacgttcaaaagcaatttccaaacgataaattattgaaaaggatgaacatcaatacttatctcgcaggcccaattacaaatccccaggatgaggtgattggcataatgactatcctgtgtaaacgcaaagtcgaatttgacacactgaataataccttgtttggtttatttttaagccgaatcaattcggagattgagagactgataaaccttagaaaattagagtttctggcgagtataccccagcaaaaccctaatccaatcataaggattcttctcaatggggatgtcgtattcactaacgaggaagggaaaatcattttaaactactggataaaattatttaagggacttccagtagagttactaaaagaagcacaaagaattcagaaatccaatcaaagtacaagaattgaaatggaaattgaggataaaatctacttattcacacttgtctggattgacgaatttaaacagatc

SEQ ID NO: 3 - DNA, 20 bp, Artificial Sequence
  Primer
ccactagttctagagcggcc

SEQ ID NO: 4 - DNA, 7 bp, Legionella pneumophila
aatattg

SEQ ID NO: 5 - DNA, 10 bp, Legionella pneumophila
gtattgtaag

SEQ ID NO: 6 - DNA, 6 bp, Legionella pneumophila
aagtag

(19) United States
(12) Patent Application Publication
(10) Pub. No.: US 2003/0165889 A1
(43) Pub. Date: Sep. 4, 2003

(54) Method of isolation of regulated determinants from bacterial pathogens

(75) Inventor: Jeffrey D. Cirillo, Lincoln, NE (US)

Correspondence Address: SENNIGER POWERS LEAVITT AND ROEDEL, ONE METROPOLITAN SQUARE, 16TH FLOOR, ST LOUIS, MO, 63102, US

(73) Assignee: The Board of Regents of the University of Nebraska

(21) Appl. No.: 10/144,907

(22) Filed: May 13, 2002

Related U.S. Application Data
(63) Continuation of application No. 09/604,561, filed on Jun. 27, 2000, now abandoned.
(60) Provisional application No. 60/141,717, filed on Jun. 30, 1999.

(51) Int. Cl. C12Q 1/68; C12Q 1/04; C12N 15/74; C12N 1/21

(52) U.S. Cl. 435/6; 435/34; 435/471; 435/252.3

(57) ABSTRACT

The present invention relates to novel methods for isolation and identification of virulence determinants from bacterial pathogens. The present invention also relates to novel genes of the Legionella pneumophila bacteria, and methods of detection of Legionella pneumophila bacteria in samples.

### CROSS-REFERENCE TO RELATED APPLICATIONS

This application is a continuation application of U.S. application Ser. No. 09/604,561, herein incorporated by reference in its entirety, which claims priority from provisional application entitled “Method for Isolation and Identification of Regulated Virulence Determinants from Bacterial Pathogens,” filed by the University of Nebraska on Jun. 30, 1999 and given serial No. 60/141,717, which is hereby incorporated by reference in its entirety. This invention was made with government support under AI40165 awarded by the National Institutes of Health. The government has certain rights to this invention.

### FIELD OF THE INVENTION

The present invention relates to the fields of microbiology and cellular biology, specifically to a new method for the isolation and identification of virulence determinants from bacterial pathogens, particularly from Legionella pneumophila, by over-expressing genes that are normally repressed under laboratory growth conditions.

### BACKGROUND OF THE INVENTION

Bacteria are often the causative agents of disease in plants, animals and humans. Pathogenic Gram-negative bacteria cause a number of pathological conditions such as bacteraemia, bacteria-related diarrhoea, pneumonia Legionnaires' disease, meningitis and (very commonly) urinary tract infections, i.a. pyelonephritis, cystitis, urethritis etc. The initiation and persistence of many bacterial infections such as those described above is thought to require the presentation of adhesins on the surface of the microbe in accessible configurations which promote binding events that dictate whether extracellular colonization, internalization or other cellular responses will occur. Adhesins are often components of the long, thin, filamentous, heteropolymeric protein appendages known as pili, fimbriae, or fibrillae. The bacterial attachment event is often the result of a stereo-chemical fit between an adhesin frequently located at the pilus tip and specific receptor architectures on host cells, often comprising carbohydrate structures in membrane associated glycoconjugates.

Other mechanisms for adherence of bacterial to the host cells have been demonstrated, including opsonization with complement and specific antibodies, including type IV pilus that can mediate non-opsonic adherence. See Gibson et al., “Adherence of Legionella pneumophila to U-937 cells, guinea-pig alveolar macrophages, and MRC-5 cells by a novel, complement-independent binding mechanism,” Can.J.Microbiol., 40:865-872 (1994); Rogers et al., “Opsonin-independent adherence and intracellular development of Legionella pneumphila within U-937 cells” Can.J.Microbiol. 39:718-722 (1993); Stone et al., “Expression of multiple pili by Legionella pneumophila: identification and characterization of type IV pilin gene and its role in adherence to mammalian and protozoan cells,” Infec.Immun. 66(4): 1768-75 (1998); Venkataraman et al., “Identification of a Gal/GalNAc lectin in the protozoan Hartmanella vermiformis as a potential receptor for attachment and invasion by the Legionnaires' disease bacterium, Legionella pneumophila,” J.Exp.Med. 186:537-547 (1997).

Infectious strains of bacteria are often hard to identify, and this can lead to mis-diagnosis and ineffective treatment of diseases. One such pathogenic bacteria is Legionella pneumophila. Legionella pneumophila is a gram-negative bacterium that is the causative agent of the potentially lethal pneumonia Legionnaires' disease. Current methods for the diagnosis of Legionnaires' disease in infected hosts are somewhat unreliable and generally result in a high level of falsely positive or falsely negative detections. Part of the problem in identifying the disease in a suspected carrier has been an inability to isolate the causative agent from the host. Successful isolation of the causative agent is required for conventional diagnosis of most bacterial infections.

Although the instances of Legionella pneumophila infection leading to outbreaks of Legionnaires' disease are relatively rare, the results of such outbreaks are quite serious. Legionnaires' disease begins with flu-like symptoms, then moves on to high fever, shaking chills, headaches, diarrhea, pneumonia, and pleurisy, and can result in death. The disease is highly contagious. The bacteria which causes this disease is only harmful when tiny droplets of water floating in the air containing the bacteria are inhaled, and does not cause harm when it is present in drinking water.

Although the Legionella pneumophila bacteria can persist for up to a year in tap water, it replicates almost exclusively intracellularly during infection and in its natural freshwater habitat. See Skaliy et al., “Survival of Legionnaries' disease bacterium in water,” Ann. Intern. Med. 90:662-663 (1979); Glavin et al., “Ultrastructure of lung in Legionnaires' disease,” Ann. Intern. Med. 90:555-559 (1979); Fields et al., “Intracellular multiplication of Legionella pneumophila in amoebae isolated from hospital hot water tanks,” Curr. Microbiol. 18:131-137 (1989). Because Legionella pneumophila must enter host cells in order to cause disease, entry into the host cell is considered to be a primary mechanism for pathogenesis of these bacteria. See Davis et al., “The kinetics of early inflammatory events during experimental pneumonia due to Legionella pneumophila in guinea pigs,” J. Infect. Dis. 148:823-825 (1983). However, exactly how entry occurs is not well understood. A better understanding of the mechanisms of pathogenesis of Legionnaires' disease is needed in order to develop better treatments for the disease.

Monocytes are the primary cell type that Legionella pneumophila replicates within during infection. See Winn et al., “The pathology of Legionella pneumophila. A review of 74 cases and the literature,” Hum. Pathol. 12:401-422 (1981). However, it has been suggested that epithelial cells may also play a role in the replication of the bacteria. Maruta et al., “Entry and intracellular growth of Legionella dimoffii in alveolar epithelial cells,” Am. J. Respir. Crit. Care Med. 157: 1967-1974 (1998); Mody et al., “Legionella pneumophila replicates within rat alveolar epithelial cells,” J. Infect. Dis. 167:1138-1145 (1993). Entry of Legionella pneumophila into monocytes has been shown to occur by an unusual mechanism termed ‘coiling phagocytosis.’ Horwitz et al., “Phagocytosis of the Legionnaires' disease bacterium (Legionella pneumophila) occurs by a novel mechanism: Engulfment within a pseudopod coil,” Cell 36:27-33 (1984). Although this asymmetric pseudopod coiling mechanism appears to be consistently observed in Legionella pneumophila and spirochetes, it is almost never or only randomly observed in other bacterial species. Rittig et al., “Coiling phagocytosis—a way fore MHC class I presentation to bacterial antigens?” Int. Arch. allerty Immunol. 103:4-10 (1994).

Among the several mechanisms of Legionella pneumophila adherence to host cells are opsonization with complement and specific antibodies including both Fc and complement receptors. See Hussmann et al., “Adherence of Legionella pneumophila to guinea pig peritoneal macrophages, J774 mouse macrophages, and undifferentiated U937 human monocytes: role of Fc and complement receptors,” Infect. Immun. 60:5212-5218 (1992); Payne et al., “Phagocytosis of Legionella pneumophila is mediated by human monocyte complement receptors,” J. Exp. Med. 166:1377-1389 (1987). Complement mediated adherence has been shown to result in phagocytosis by monocytes and is thought to involve the Legionella pneumophila major outer membrane protein (MOMP). However, no specific Legionella pneumophila mutants have been constructed in this gene, which prevents elucidation of its exact role in entry. Potential host cell receptors for non-opsonic adherence have been suggested and a type IV pilus that can mediate this type of adherence has been described. However, in the absence of this pilus Legionella pneumophila adheres at 53% of the wild-type levels, suggesting the presence of multiple adherence mechanisms.

There is a need for a reliable method of identifying additional bacterial factors involved in the entry of L. pneumophila in to host cells, as well as a need for a method of identification of other regulated virulence determinants from a wide range of bacterial pathogens.

### SUMMARY OF THE INVENTION

Accordingly, it is an object of the present invention to provide a method of isolating and identifying bacterial genes that relate to virulence determinants comprising obtaining a pool of mutants of bacteria with an enhanced virulence phenotype, selecting a mutant bacteria from the pool of mutants that displays the enhanced virulence phenotype, locating the DNA of the mutant bacteria that contains a sequence that is a factor in the enhanced virulence phenotype, inserting random mutations in the DNA that contains the sequence that is a factor in the enhanced virulence phenotype, transferring the DNA into wild-type bacteria, selecting the bacteria that no longer express the enhanced virulence phenotype in order to identify an active site of the DNA necessary to confer the enhanced virulence phenotype to the wild-type bacteria, inserting specific mutations in those regions of the identified DNA, transferring the DNA with specific mutations into the wild-type bacteria, and comparing the resulting phenotype of the mutant bacteria with the specific mutations to the wild-type bacteria to identify the loci of genes responsible for the enhanced virulence phenotype.

Another aspect of the invention is a rtxA gene of Legionella pneumophila of the sequence of SEQ. ID. NO. 1 or complement thereof. Also, the present invention relates to a non-chromosome nucleic acid molecule having a sequence selected so that the nucleic acid molecule specifically hybridizes under conditions of high stringency to a rtxA gene or an mRNA encoded by a rtxA gene. This invention also relates to a kit for detecting or quantitating Legionella pneumophila comprising a container holding at least one nucleic acid molecule as described above.

Yet another aspect of the invention is an enhC gene of Legionella pneumophila of SEQ. ID. NO. 2 or complement thereof. Also, the present invention relates to a non-chromosome nucleic acid molecule having a sequence selected so that the nucleic acid molecule specifically hybridizes under conditions of high stringency to a enhC gene or an mRNA encoded by a enhC gene. This invention also relates to a kit for detecting or quantitating Legionella pneumophila comprising a container holding at least one nucleic acid molecule as described above.

Additionally, the present invention relates to a method of detection of Legionella pneumophila comprising obtaining DNA from a sample suspected of containing Legionella pneumophila, amplifying the DNA by polymerase chain reaction using as primers a plurality of nucleic acid molecules according to those discussed above having a sequence selected so that the nucleic acid molecule specifically hybridizes under conditions of high stringency to a enhC gene or a rtxA gene or an mRNA encoded by a enhC gene or by a rtxA gene, and detecting or quantitating the Legionella pneumophila by detecting or quantitating the amplified DNA.

Yet another aspect of the invention is a method of detecting or quantitating Legionella pneumophila comprising obtaining a DNA or RNA from a sample suspected of containing L. pneumophila, contacting the DNA or RNA with a nucleic acid molecule having a sequence selected so that the nucleic acid molecule specifically hybridizes under conditions of high stringency to a enhC gene or a rtxA gene or an mRNA encoded by a enhC gene or by a rtxA gene, so that the molecule hybridizes to the DNA or RNA, and detecting or quantitating the L. pneumophila by detecting or quantitating the nucleic acid molecule hybridized to the DNA and RNA.

Moreover, the present invention relates to a method of detecting or quantitating Legionella pneumophila comprising obtaining a sample suspected of containing Legionella pneumophila antigens, contacting said sample with antibody against the protein product of the RtxA gene or of the enhC gene or complements thereof, and detecting or quantitating the antigen by detecting or quantitating the antibody bound to the antigen.

Furthermore, the present invention relates to a method of diagnosis of Legionnaires' disease in a subject comprising obtaining DNA from a subject, amplifying the DNA by polymerase chain reaction using as primers a plurality of nucleic acid molecules according to those discussed above having a sequence selected so that the nucleic acid molecule specifically hybridizes under conditions of high stringency to a enhC gene or a rtxA gene or an mRNA encoded by a enhC gene or by a rtxA gene, and detecting or quantitating the Legionella pneumophila by detecting or quantitating the amplified DNA.

In yet another aspect of the present invention is a method of diagnosis of Legionnaires disease in a subject comprising obtaining a DNA or RNA from a sample suspected of containing L. pneumophila, contacting the DNA or RNA with a nucleic acid molecule having a sequence selected so that the nucleic acid molecule specifically hybridizes under conditions of high stringency to a enhC gene or a rtxA gene or an mRNA encoded by a enhC gene or by a rtxA gene, so that the molecule hybridizes to the DNA or RNA, and detecting or quantitating the L. pneumophila by detecting or quantitating the nucleic acid molecule hybridized to the DNA and RNA.

Another aspect of the present invention is a method of diagnosis of Legionella pneumophila infection in a subject comprising obtaining a sample from a subject suspected of containing Legionella pneumophila antigens, contacting said sample with antibody against the protein product of the RtxA gene or of the enhC gene or complements thereof, and detecting or quantitating the antigen by detecting or quantitating the antibody bound to the antigen.

Additionally, the present invention relates to a method of treatment of Legionella pneumophila infection in a subject comprising designing an inhibitor of the activities of the product of either the rtxA gene or the enhC gene or complements thereof, producing said inhibitor, and administering said inhibitor to said subject.

The present invention further relates to a method of treatment of Legionella pneumophila infection in a subject comprising designing compounds that block the ability of the product of the rtxA or of the enhC gene to bind to host cells, producing these compounds that block said binding to host cells, and administering the compounds to the subject.

Moreover, the present invention relates to a method of prevention of Legionella pneumophila infection in a subject comprising producing the protein product of either the rtxA gene or of the enhC gene, purifying the protein, and vaccinating the subject with the protein. Another method of prevention of Legionella pneumophila infection in a subject comprises cloning the rtxA gene or the enhC gene into an appropriate organism, allowing the organism to express either the rtxA gene or the enhC gene, and vaccinating the subject with the organism.

Another aspect of the present invention is the method of prevention of Legionella pneumophila infection in a subject comprising cloning the rtxA gene or the enhC gene into a eukaryotic expression plasmid, and vaccinating the subject with the plasmid. Additionally, the present invention relates to a method of prevention of Legionella pneumophila infection in a subject comprising constructing a mutation in the rtxA gene or in the enhC gene, introducing the mutation into Legionella pneumophila and vaccinating the subject with the mutated Legionella pneumophila.

Abbreviations and Definitions

To facilitate understanding of the invention, a number of terms as used herein are defined below:

As used herein, the terms “enhanced entry phenotype” relates to the phenotype of certain mutant bacteria to enter into a host cell at a higher frequency than entry into the host cell by the wild-type bacteria.

As used herein, the terms “adherence” relates to the ability of a bacteria to attach to a host cell.

As used herein, the terms “virulence determinants” relates to the degree of pathogenicity of a bacteria as indicated by its ability to infect a mammalian subject.

As used herein, the terms “enhanced virulence phenotype” relates to the phenotypes of bacteria for adherence, entry, intracellular growth, intracellular replication, cytotoxicity and replication of the bacteria in a mammalian subject.

As used herein, the terms “EMS mutagenesis” shall mean ethylmethane sulfonate mutagenesis.

As used herein, the term “plating” shall mean the cultivation of microorganisms on a solid nutrient medium in a petri dish.

As used herein, the term “ORF” shall mean open reading frame, which is a segment in mRNA that contains codons that can be translated into an amino acid sequence and that does not contain a termination codon.

As used herein, the term “rtxA” gene is a L. pneumophila gene that hybridizes to the rtxA gene or a fragment thereof under conditions of high stringency. High stringency conditions are defined herein to be those conditions giving hybridization only if there are about 10% or less base pair mismatches. The sequence of the coding region of the rtxA gene of the AA100 strain and portions of the flanking regions are given in FIG. 18 and the sequence is identified in SEQ ID NO. 1. RtxA genes other than rtxA gene of strain AA100 can be identified and isolated using methods well known in the art. For instance, probes based on the sequence of the rtxA gene of strain AA100 can be used to screen L. pneumophila genomic libraries for other rtxA genes. The term “rtxA” gene also includes a polynucleotide that has at least 90% sequence identity with SEQ. ID. NO. 1 and encodes a protein having the same biological function as the protein encoded by SEQ. ID. NO. 1.

As used herein, the term “enhC” gene is a L. pneumophila gene that hybridizes to the enhC gene or a fragment thereof under conditions of high stringency. High stringency conditions are defined herein to be those conditions giving hybridization only if there are about 10% or less base pair mismatches. The sequence of the coding region of the enhC gene of the AA100 strain and portions of the flanking regions are given in FIG. 19 and the sequence is identified in SEQ ID NO. 2. enhC genes other than enhC gene of strain AA100 can be identified and isolated using methods well known in the art. For instance, probes based on the sequence of the enhC gene of strain AA100 can be used to screen L. pneumophila genomic libraries for other enhC genes. The term “enhC” gene also includes a polynucleotide that has at least 90% sequence identity with SEQ. ID. NO. 2 and encodes a protein having the same biological function as the protein encoded by SEQ. ID. NO. 2.

As used herein, the terms “Legionella pneumophila” and “L. pneumophila” are used interchangeably to mean the bacteria that exists in many environments such as human alveolar macrophages, fresh water protozoa, and biofilms. These bacteria are the agent of Legionnaires' disease.

As used herein, the terms “random integrating controlled expression” and “RICE” shall mean a type of DNA library which consists of isolated total DNA of a bacteria placed in a plasmid that does not replicate in the bacteria of interest and carries a regulated or not regulated promoter that functions in the bacteria of interest and is located immediately upstream of the site for insertion of the isolated DNA. This plasmid DNA library is then transferred into the wild-type bacteria for the purpose of creating a mutant of the wild-type bacteria.

As used herein, the term “a.a.” shall mean amino acid, which is an organic compound that is the building block of peptides and proteins.

As used herein, the term “enh” relates to the enhanced virulence phenotype.

As used herein, the term “b.p.” shall mean base pair, which is a unit of length in a nucleic acid molecule.

### DESCRIPTION OF THE FIGURES

FIG. 1A is a graph representing the results from a representative selective entry assay showing the ability of an individual EMS mutagenized L. pneumophila clone to enter into epithelial cells after a selective entry assay. Data points and error bars represent the mean of triplicate samples from a representative experiment and their standard deviations. All experiments were performed at least twice.

FIG. 1B is a graph representing the results from a representative selective entry assay showing the ability of an individual EMS mutagenized L. pneumophila clone to enter into monocytes after a selective entry assay. Data points and error bars represent the mean of triplicate samples from a representative experiment and their standard deviations. All experiments were performed at least twice.

FIG. 2A is a graph representing the results from a selective entry assay showing the entry into epithelial cells of wild-type (AA100) L. pneumophila and AA100 transformed with the parent cosmid vector (pYUB289)or cosmids from a total genomic DNA library of enhanced entry mutant C3. Data points and error bars represent the mean of triplicate samples from a representative experiment and their standard deviations. All experiments were performed at least twice. The * symbol indicates those cosmid clones that were selected for further analysis.

FIG. 2B is a graph representing the results from a selective entry assay showing the entry into monocytes of wild-type (AA100) L. pneumophila and AA100 transformed with the parent cosmid vector (pYUB289)or cosmids from a total genomic DNA library of enhanced entry mutant C3. Data points and error bars represent the mean of triplicate samples from a representative experiment and their standard deviations. All experiments were performed at least twice.

FIG. 3A and FIG. 3B are graphs representing the results from an adherence assay showing adherence to epithelial cells in FIG. 3A and to monocytes in FIG. 3B of wild-type (A100) L. pneumophila and A100 transformed with the parent cosmid vector (pYUB289) or cosmids from a total genomic DNA library of enhanced entry mutant C3. Data points and error bars represent the mean of triplicate samples from a representative experiment and their standard deviations. All experiments were performed at least twice.

FIG. 4 shows the results of determination of loci responsible for enhanced entry phenotype by transposon mutagenesis of cosmids 1A3 and 2A6. The position of each unique transposon insertion is indicated. The symbols indicate transposon insertions that result in cosmids that confer levels of entry to AA100 that are (−) or are not (+) significantly different from that of the parent cosmid (1A3 or 2A6).

FIG. 5 is a graph representing the results of selective entry assays for wild-type AA100 L. pneumophila, and AA-100 containing cosmid 1A-3, and AA-100 containing cosmids pJDC19, pJDC20, pJDC23, and pJDC24 (all for enh1 and enh2 loci for enhanced entry phenotype). Data points and error bars represent the mean of triplicate samples from a representative experiment and their standard deviations. All experiments were performed at least twice.

FIG. 6 is a sequence analysis of enh1 and enh2 loci. Gene designation and the direction of transcription of each of the ORFs found within these loci are shown directly below the construct. The size of each locus is shown in parentheses.

FIG. 7A is a diagram showing characteristics of the L. pneumophila rtxA gene. Locations of putative translational start, stop, and ribosomal binding site sequences are indicated. Open boxes indicate the positions of individual nine amino acid RTX repeat sequences within the coding region of rtxA.

FIG. 7B is a diagram showing alignment of the 64 amino acids highly conserved region including the three carboxy terminal RTX repeat sequences. Bold indicates residues that are conserved in the majority of aligned sequences. Numbers adjacent to the alignment indicate the positions of the aligned amino acids within the coding sequence of the RTX proteins. Abbreviations for the aligned RTX repeat regions are: LpRtxA.f1=L. pneumophila amino terminal region; LpRtxA.f2=L. pneumophila carboxy terminal region; BbClyA=B. pertussis CyaA; EcHlyA=E. coli HlyA; AcClyIIA=Actinobacillus pleuropneumoniae ClyIIA; AnHlyA=Anabaena sp. strain PCC 7120 HlyA; NeFrpA=Neisseria meningitidis FrpA; and PaHla1=Pasteurella haemolytica Hla1.

FIG. 8A is a diagram showing characteristics of the L. pneumophila enhA, enhB and enhC genes. Locations of putative translational start, stop, and ribosomal binding site sequences are indicated. Open boxes indicate the positions of putative secretory signal sequences, the box filled with a grid indicates the TPR domain and boxes filled with diagonals indicate the two highly conserved regions.

FIG. 8B is a diagram showing the alignment of the 154 amino acid conserved region with similar regions of other proteins. Bold indicates residues that are conserved in the majority of aligned sequences. Numbers adjacent to the alignment indicate the position of the aligned amino acids within the coding sequence of the proteins. Abbreviations for the aligned conserved region are: LpEnhC.d1=L. pneumophila amino terminal conserved region; LpEnhC.d2=L. pneumophila carboxy terminal conserved region; CeSel1=C. elegans Sel-1; CaCsr=C. albicans chitin synthase regulatory factor; ScSkt5=S. cerevisiae Skt5.

FIG. 9 is diagram showing the structure of the rtxA region, ΔrtxA mutant and complementing constructs. All constructs are in single-copy in the L. pneumophila chromosome except pJDC20, which is present in the low-copy number plasmid pYUB289. The ΔrtxA mutant is an in-frame deletion in the rtxA gene producing a 130 a.a. protein product, 6 a.a. from the amino terminus and 124 a.a. from the carboxy terminus. Gene designations are below the arrows on the constructs illustration the direction of transcription of the genes. Open boxes in AA100 indicate the positions of the nine amino acid repeats sequences characteristic of RTX proteins.

FIG. 10(A) is a graph showing the ability of AA100, the ΔrtxA mutant and complemented clones to adhere to HEp-2 epithelial cells. Data points and error bars represent the mean of triplicate samples from a representative experiment and their standard deviations. All experiments were performed at least three times.

FIG. 10(B) is a graph showing the ability of AA100, the ΔrtxA mutant and complemented clones to adhere to THP-1 monocytic cells. Data points and error bars represent the mean of triplicate samples from a representative experiment and their standard deviations. All experiments were performed at least three times.

FIG. 11(A) is a graph showing the ability of AA100,the ΔrtxA mutant and complemented clones to adhere to formaldehyde fixed HEp-2 epithelial cells. Data points and error bars represent the mean of triplicate samples from a representative experiment and their standard deviations. All experiments were performed at least three times.

FIG. 11(B) is a graph showing the ability of AA100,the ΔrtxA mutant and complemented clones to adhere to formaldehyde fixed THP-1 monocytic cells. Data points and error bars represent the mean of triplicate samples from a representative experiment and their standard deviations. All experiments were performed at least three times.

FIG. 12 is a graph representing the cytotoxicity of AA100, the ΔrtxA mutant and ΔrtxA transformed with pJDC20, pJDC35, and pJDC40 for human monocytic cell line THP-1. Data points and error bars represent the mean of triplicate samples from a representative experiment and their standard deviations. All experiments were performed at least three times.

FIG. 13(A)is a graph representing pore formation of AA100, the ΔrtxA mutant and ΔrtxA transformed with pJDC20, pJDC35, and pJDC40 for THP-1 cells. Data points and error bars represent the mean of triplicate samples from a representative experiment and their standard deviations. All experiments were performed at least three times.

FIG. 13(B) is a graph representing pore formation of AA100, the ΔrtxA mutant and ΔrtxA transformed with pJDC20, pJDC35, and pJDC40 for U-937 cells. Data points and error bars represent the mean of triplicate samples from a representative experiment and their standard deviations. All experiments were performed at least three times.

FIG. 13(C) is a graph representing pore formation of AA100, the ΔrtxA mutant and ΔrtxA transformed with pJDC20, pJDC35, and pJDC40 for RAW264.7 cells. Data points and error bars represent the mean of triplicate samples from a representative experiment and their standard deviations. All experiments were performed at least three times.

FIG. 13(D) is a graph representing pore formation of AA100, the ΔrtxA mutant and ΔrtxA transformed with pJDC20, pJDC35, and pJDC40 for J774.1 cells. Data points and error bars represent the mean of triplicate samples from a representative experiment and their standard deviations. All experiments were performed at least three times.

FIG. 14(A) is a graph showing the growth of AA100 and the ΔrtxA mutant in BYE as measured by optical density at 600 nm. Data points and error bars represent the mean of triplicate samples from a representative experiment and their standard deviations. Error bars are not visible since they overlap with the symbols. All experiments were performed at least three times.

FIG. 14(b) is a graph showing the growth of AA100 and the ΔrtxA mutant in BYE as measured by colony forming units (cfu). Data points and error bars represent the mean of triplicate samples from a representative experiment and their standard deviations. Error bars are not visible since they overlap with the symbols. All experiments were performed at least three times.

FIG. 15 is a graph showing growth of AA100, the ΔrtxA mutant and ΔrtxA transformed with pJDC20, pJDC35, and pJDC40 in THP-1 cells over 48 hours. Data points and error bars represent the mean number of colony forming units (cfu) present at 48 hours/number of cfu present at time zero (Mean CFU Tx/To) of triplicate samples from a representative experiment and their standard deviations. All experiments were performed at least three times.

FIG. 16(A) is a graph showing the growth of AA100 and the ΔrtxA mutant in THP-1 monocytic cells during the first 24 hours after entry. Data points and error bars represent the mean of triplicate samples from a representative experiment and their standard deviations. Many of the error bars are not visible since they overlap with the symbols. All experiments were performed at least three times.

FIG. 16(b) is a graph showing the growth of AA100 and the ΔrtxA mutant in THP-1 monocytic cells during the first 48 hours after entry. Data points and error bars represent the mean of triplicate samples from a representative experiment and their standard deviations. Many of the error bars are not visible since they overlap with the symbols. All experiments were performed at least three times.

FIG. 17 is an image showing histopathologic examination of mouse lungs at 48 hours after infection with wild-type (B-D) and the rtxA mutant (A) L. pneumophila strains. Lung sections were stained with hematoxylin and eosin (A,B) or Warthin-Starry silver (C,D). A characteristic example of a mouse lung infected with wild-type L. pneumophila (B) displaying a severe peribronchial pneumonia with leukocytic exudate, necrotic debris and red blood cells within two small bronchioles. In striking contrast, lungs infected with the ΔrtxA mutant (A) displayed similar characteristics to that of normal healthy mouse lungs, with clear bronchioles and alveoli showing very little or no inflammatory infiltrate. Silver stained sections (C,D) allowed visualization of bacteria (arrows), apparently located as clusters intracellularly in mononuclear cells. The magnification of images is 400× (A,B) and 1000× (C,D).

FIG. 18 is a sequence listing of the rtxA gene as identified in SEQ ID NO. 1.

FIG. 19 is a sequence listing of the enhC gene as identified in SEQ ID NO. 2.

### DETAILED DESCRIPTION OF THE INVENTION

All publications, patents, patent applications and other references cited in this application are herein incorporated by reference in their entirety as if each individual publication, patent, patent application or other reference were specifically and individually indicated to be incorporated by reference.

Applicants have discovered a novel method of isolation and identification of bacterial genes by inducing an over-expression of the gene of interest in order to directly select for the gene of interest, particularly genes related to virulence determinants.

The present invention is directed to methods of isolating and identifying genes related to virulence determinants in bacteria. The method involves selecting mutants of a bacteria, which express the phenotype of interest, as opposed to the wild-type of the same bacteria. In a preferred embodiment, mutation of the wild-type of the bacteria of interest is done with chemical methods known in the art in order to produce the mutant bacteria with the phenotype of interest. In a more preferred embodiment, mutation of the bacteria of interest is done with ethylmethane sulfonate (EMS). See Miller, J. H. Experiments in Molecular Genetics, Cold Spring Harbor, N.Y. (1972). In another preferred embodiment, mutation of the bacteria of interest is done with transposon mutagenesis. See Alexeyev et al., “Mini-Tn10 transposon derivatives for insertion mutagenesis and gene delivery into the chromosome of Gram-negative bacteria,” Gene 160:9-62 (1995). Furthermore, in other preferred embodiments, the mutants of the bacteria of interest are created using a random integrating controlled expression library, or a library constructed in a replicating plasmid.

The method of the present invention also requires a selection strategy for the purpose of selecting the bacteria with the phenotype of interest. In order to select a bacteria with an over-expression of the gene sought to be identified, a positive selection strategy is required. In order to select a bacteria with a reduced-expression of the gene sought to be identified, a negative selection strategy is required. In a preferred embodiment, the selection strategy is through the use of entry and/or adherence assays to determine the effective level of entry of a bacteria into a host cell, or the effective level of adherence of a bacteria to a host cell. See Cirillo, et al., “Growth of Legionella pneumophila in Acanthamoeba castellanti enhances invasion,” Infect. Immun. 62:3254-3261 (1994). In another preferred embodiment, the selection strategy is through the use of cytotoxicity assays. See Behl et al., “Hydrogen peroxide mediates amyloid beta protein toxicity,” Cell 77(6):817-27 (1994); Brander et al., “Carrier-mediated uptake and presentation of major histocompatibility complex class I-restricted peptide,” Eur.J.Immunol. 23(12):3217-23 (1993). More preferably the cytotoxicity assay is a standard lactate dehydrogenase (LDH) release cytotoxicity assay. In yet another preferred embodiment, the selection strategy is through the use of pore-formation assays. See Kirby et al., “Evidence for pore forming ability by Legionella pneumphila,” Mol.Microbiol. 27:323-336 (1998); Zuckman et al., “Pore-forming activity is not sufficient for Legionella pneumophila phagosome trafficking and intracellular growth,” Molec.Microbiol. 32:990-1001 (1999). More preferably the pore-formation assay is done by ethidium bromide and acridine orange staining techniques known in the art. In another preferred embodiment, the selection strategy is through the use of intracellular growth assays with methods known in the art. See Zuckman et al., “Pore-forming activity is not sufficient for Legionella pneumophila phagosome trafficking and intracellular growth,” Molec.Microbiol. 32:990-1001 (1999).

The present invention requires the use of a host cell for determination of the virulence phenotype of the bacteria of interest. Any mammalian cell may be used as a host cell. In addition, protozoan hosts for the bacteria may also be used. In a preferred embodiment, the host cell consists of epithelial cells or monocytes. More preferrably, the host cell is a human monocyte or epithelial cell. Additionally, the culture media useful for growing the bacteria and host cells of this invention are well known in the art and include BCYE agar, Lennox broth agar, and RPMI 1640. See Edelstein et al., “Improved semiselective medium for isolation of Legionella pneumophila fromcontaminated clinical and environmental specimens,” J.Clin.Microbiol. 14:298-303 (1981). Antibiotics are used with the culture medium for the purpose of eliminating those bacteria without the acquired antibiotic resistance. Some examples of antibiotics that may be used are gentamicin, kanamycin and amikacin. In a preferred embodiment, the dosage of these antiboitics is approximately 100 μg/ml. The washing of the host cells performed during the various assays may be done with any appropriate wash solution, such as phosphate buffered saline (PBS).

In order to locate the portions of DNA that contain at least one sequence that is a factor in the enhanced virulence phenotype of the mutated bacteria exhibiting the enhanced virulence phenotype, a comparison of the DNA of both the wild-type bacteria and the mutant bacteria is made. The complete DNA sequences of both the wild-type bacteria and the mutant bacteria are determined by methods known in the art, including the use of cosmid libraries and sequence analysis using a DNA primer and automated sequencing apparatus. Comparison of the DNA sequences of the mutant bacteria and the wild-type bacteria to determine the portions of the DNA of the mutant bacteria that contain at least one sequence that is a factor in the enhanced virulence phenotype are done with methods known in the art. See Altschul et al., “Gapped BLAST and PSI-BLAST: a new generation of protein database search programs,” Nuc. Acids Res. 25:3389-3402 (1997).

The method of the present invention also requires the use of random mutations into the identified DNA portions of the mutant bacteria in order to determine the active sites of these portions of DNA for the purpose of expression of the enhanced virulence phenotypes. The use of transposon mutagenesis is helpful for the identification of gene loci because a transposon insertion into a gene loci responsible for enhanced virulence phenotype will negate the ability of that gene loci to confer the enhanced virulence phenotype. In a preferred embodiment, cosmids carrying the bacterial genomic fragments are mutagenized with a transposon carried on a plasmid. In a more preferred embodiment, the mini-Tn10 transposon is used with methods known in the art. See Alexeyev et al., “Mini-Tn10 transposon derivatives for insertion mutagenesis and gene delivery into the chromosome of Gram-negative bacteria,” Gene 160:9-62 (1995). An example of a plasmid that may be used to carry such a transposon is pKV32. See Visick et al., “New genetic tools for use in the marine bioluminescent bacterium,” Vibrio fisheri, p. 119-122. In J. W. Hastings et al. (ed.), Bioluminescence and Chemiluminescence. Proceedings of the 9th International Symposium, John Wiley and Sons Ltd., Chichester, England 1996. After the multiple random transposon insertions, the sites of the transposon insertions into the cosmids are determined using restriction analysis methods known in the art. For each of the transposon insertions, the ability to confer the enhanced virulence phenotype can be tested by direct electroporation of resulting plasmids into the wild-type bacteria and testing in the same manner as the original isolation of the mutagenized enhanced virulence mutants discussed above. See Dower et al., “High efficiency transformation of E.Coli by high voltage elctroporation,” Nucleic Acids Res. 16(13):6127-6145 (1988).

After the regions of DNA of the bacteria are identified by the methods discussed above, specific known mutations may be introduced into the wild-type DNA to determine the role of the DNA in virulence. Techniques for mutagenesis that are well known in the art include insertional mutagenesis, transposon mutagenesis, deletional mutagenesis, in-frame deletional mutagenesis, etc. See Zuckman et al., “Pore-forming activity is not sufficient for Legionella pneumophila phagosome trafficking and intracellular growth,” Molec.Microbiol. 32:990-1001 (1999). Introduction into wild-type is carried out with methods known in the art. See Zuckman et al., “Pore-forming activity is not sufficient for Legionella pneumophila phagosome trafficking and intracellular growth,” Molec.Microbiol. 32:990-1001 (1999). In addition, any DNA that is involved in Legionella pneumophila virulence may be used to treat and prevent Legionella pneumophila infection. For example, infection can be treated by administering a pharmaceutically effective amount of an oligonucleotide that inhibits the mRNA activity of Legionella pneumophila. These could be prepared from the DNA sequences described in the present invention.

In addition, compounds which bind to the protein products of these DNA regions can be produced and administered. These compounds would inhibit the activity of the protein product. Because these products are required for the bacteria to enter host cells, the inability of the bacteria to enter host cells in the presence of these compounds can be used to measure their activity.

Compounds that inhibit the activity of these protein products can be produced by overexpressing the protein, purifying the overexpressed protein, performing x-ray crystallography on the purified enzyme so as to obtain the molecular structure of the enzyme, and then creating a compound with a similar molecular structure to the protein. This compound can be administered so as to inhibit the activity of the protein, thereby preventing entry into host cells. The methods used to develop appropriate compounds are well known in the art. See Dessen et al., “Crystal structure and function of the isoniazid target of Mycobacterium tuberculosis” Science 267:1638-1641 (1995). Alternatively, combinatorial chemistry may be used to randomly generate compounds. These compounds could then be tested for their ability to inhibit the activity of the protein, thereby preventing entry into host cells. Those compounds that prevent entry of Legionella pneumophila into host cells could be administered as a treatment for Legionella pneumophila infection. The methods used to develop appropriate compounds are well known in the art. See Barry et al., “Use of genomics and combinatorial chemistry in the development of new antimycobacterial drugs” Biochem. Pharmacol. 59:221-231 (2000).

In addition, vaccines useful in the treatment and transmission prevention of Legionella pneumophila infection can be produced. Because the inventors have determined the sequence of regions of DNA involved in the ability to cause disease, it is possible to determine the existence of mutated Legionella pneumophila DNA in this region. A Legionella pneumophila bacterium mutated in this DNA can be administered in vaccine form to treat and prevent Legionella pneumophila infection. The methods used to produce these vaccines are well known in the art. See Benyacoub et al., “Attenuation and immunogenicity of Deltacya Deltacrp derivatives of Salmonella cholerasuis in pigs,” Infect. Immun. 67:3674-3679 (1999).

In addition, vaccines can be formed which comprise a Legionella pneumophila strain having a deletion in DNA in this region. This bacterial strain may then be administered in vaccine form. Alternatively, a recombinant vaccine candidate organism such as vaccinia virus or Mycobacterium bovis (BCG) containing this region of DNA could be constructed. This recombinant organism may then be administered in vaccine form. The methods used to produce recombinant vaccines are well known in the art. See Cirillo et al., “Bacterial vaccine vectors and bacillus Calmette-Guerin,” Clin. Infect. Dis. 20:1001-1009 (1995). Alternatively, this DNA region may be cloned into an expression vector that may be used for DNA vaccines. This recombinant DNA molecule may then be administered in vaccine form. The methods used to produce DNA vaccines are well known in the art. See Lowrie et al., “Therapy of tuberculosis in mice by DNA vaccination,” Nature 400:269-271 (1999).

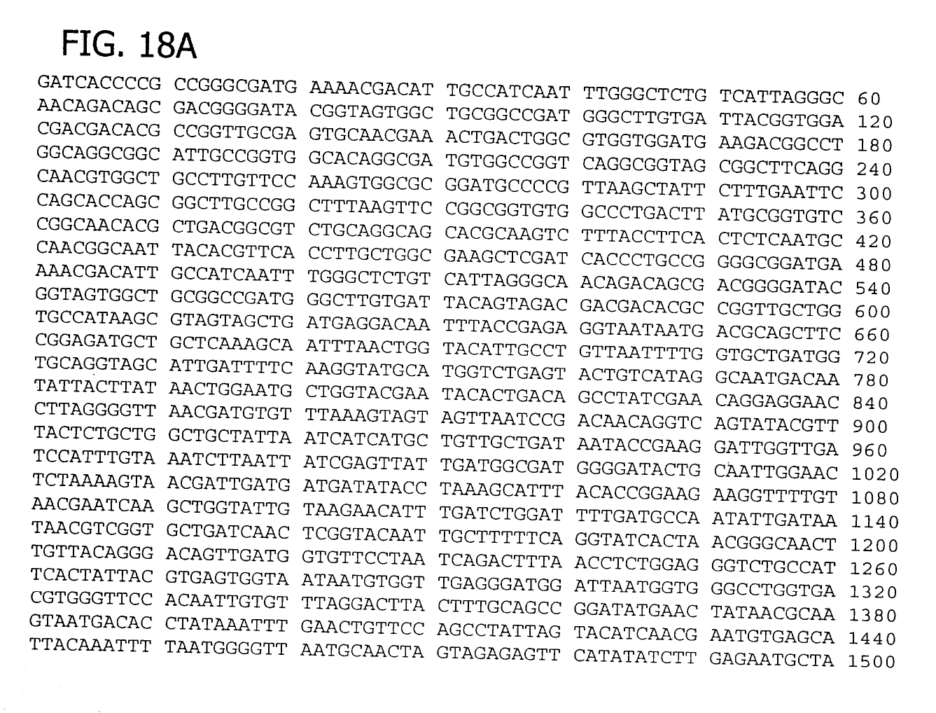

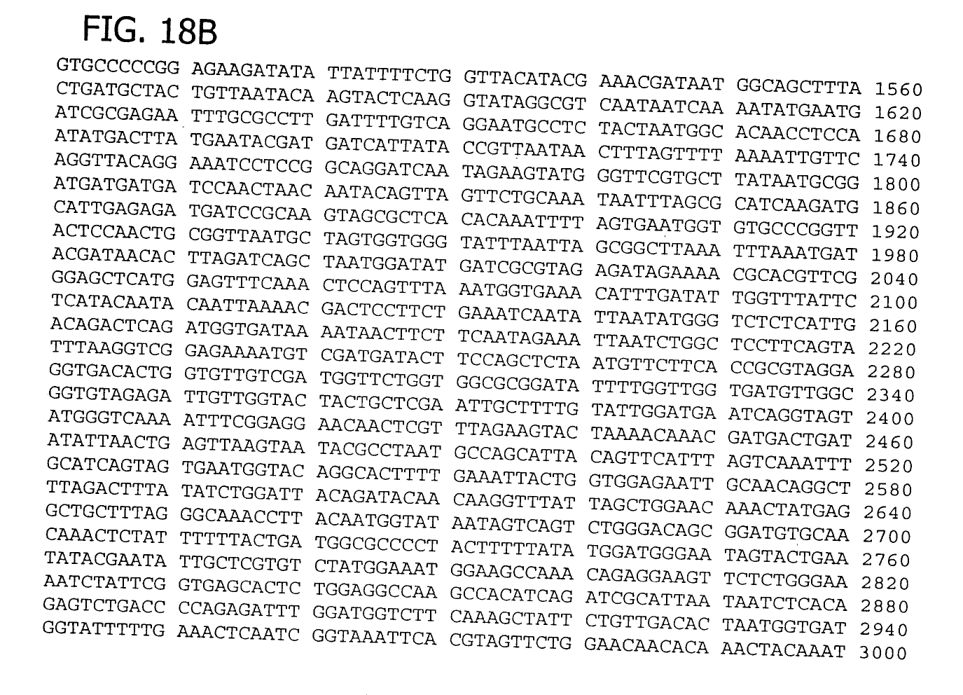

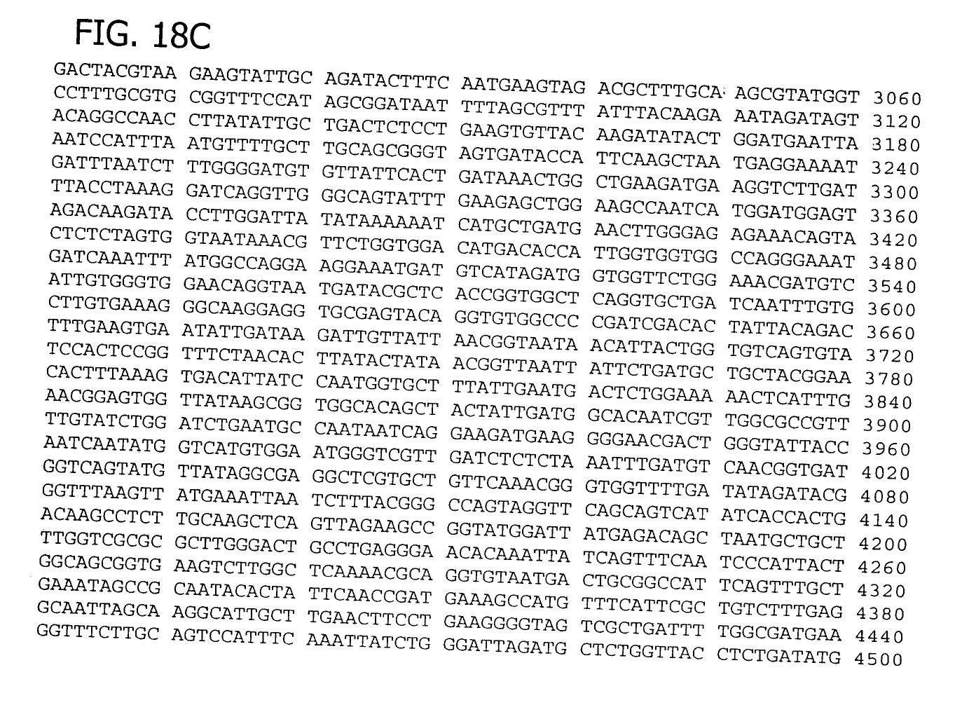

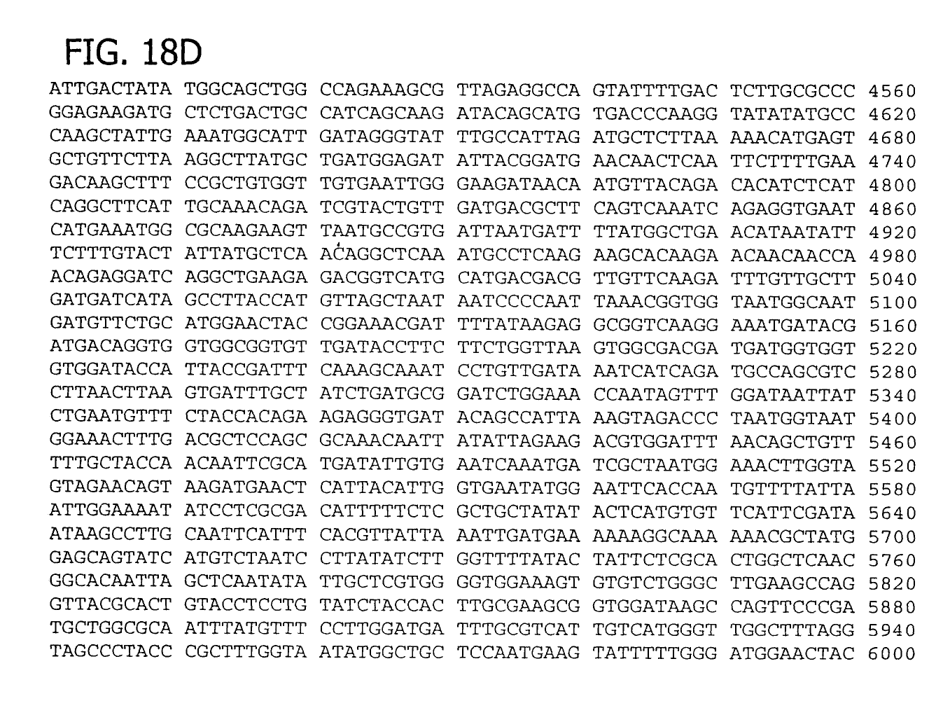

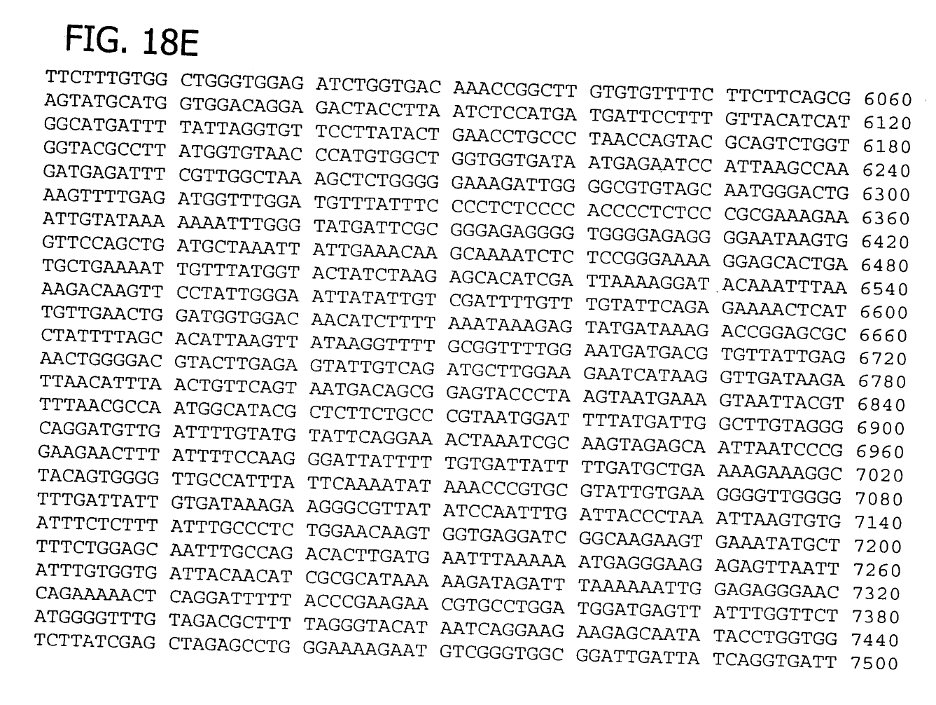

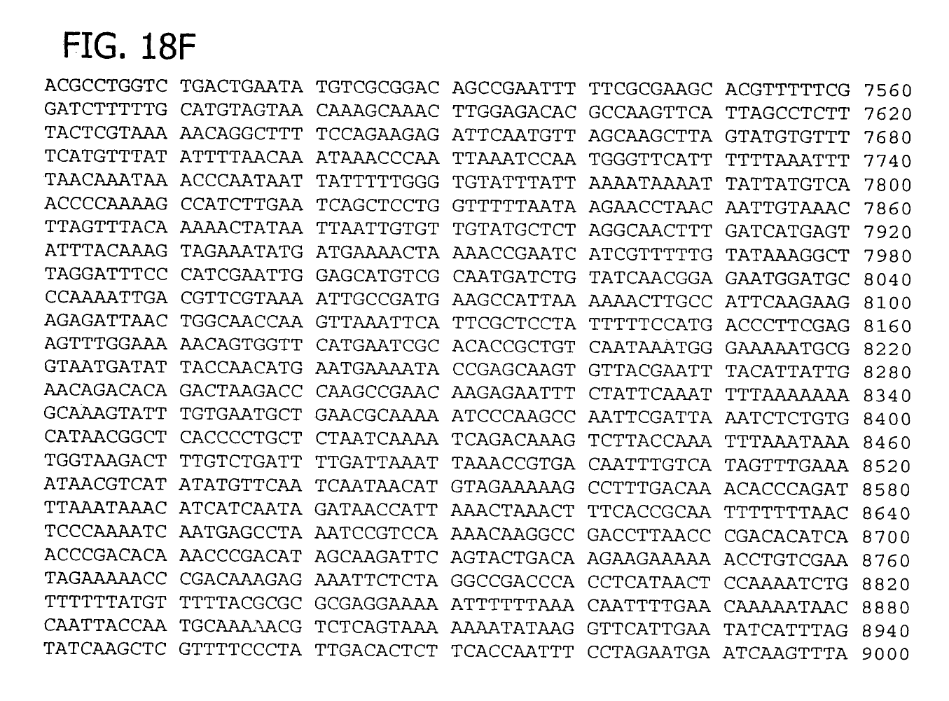

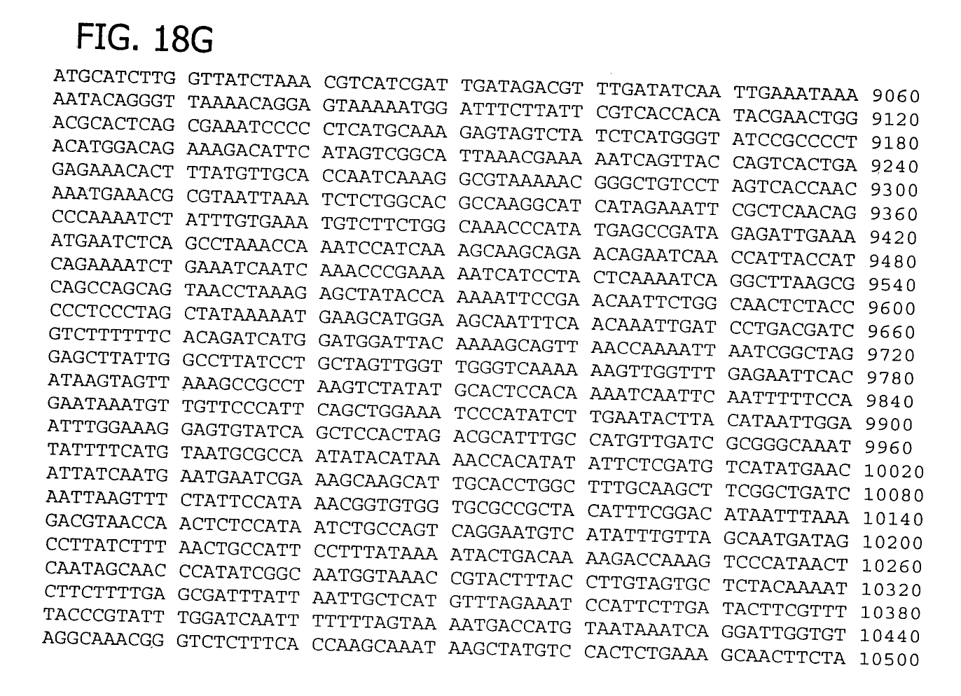

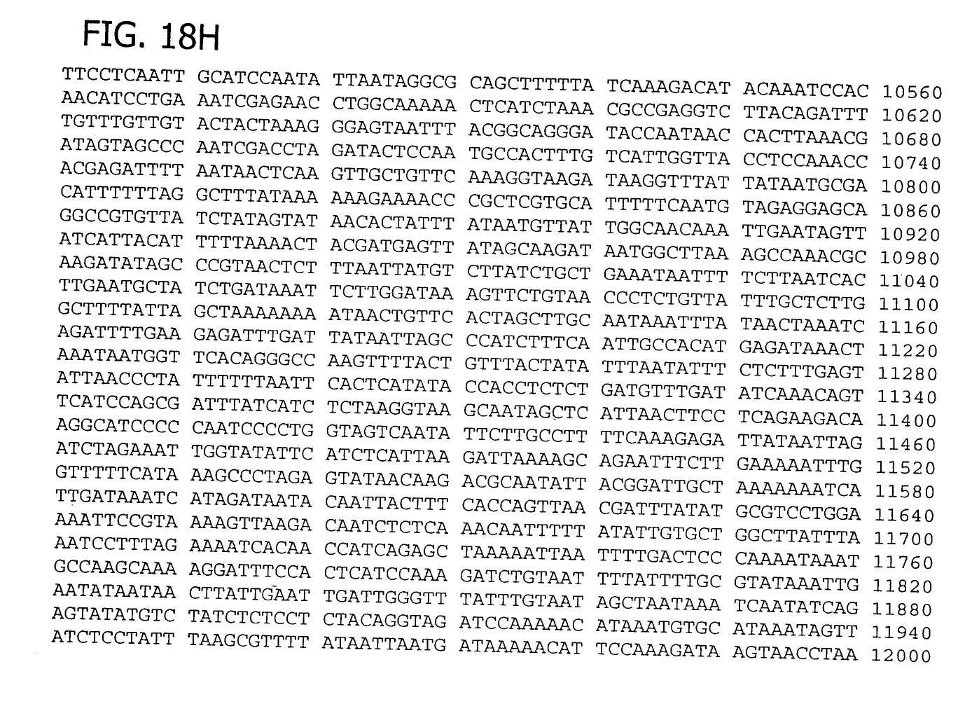

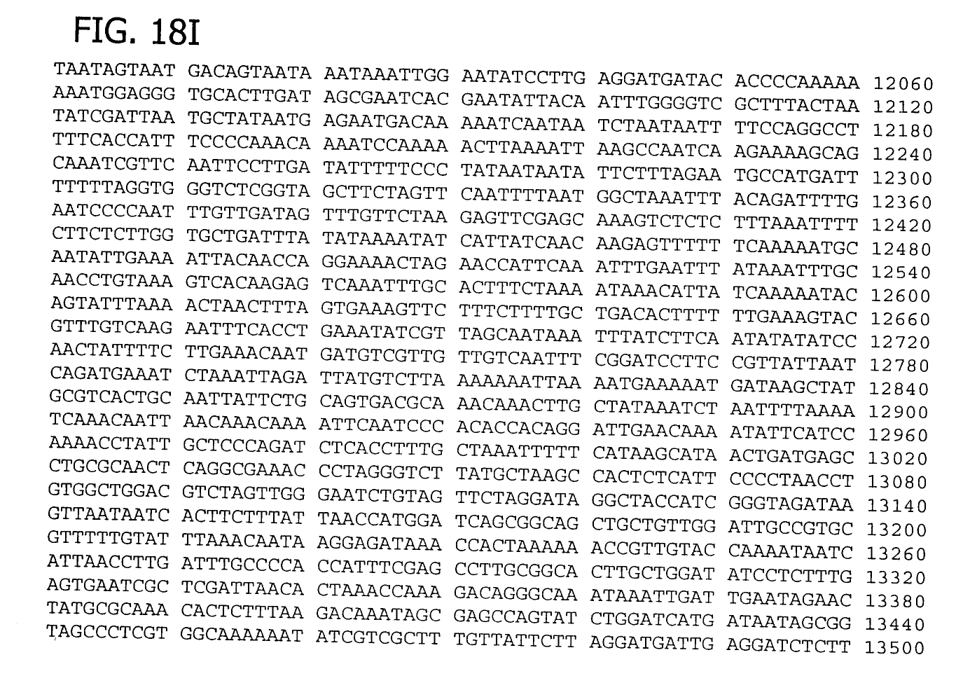

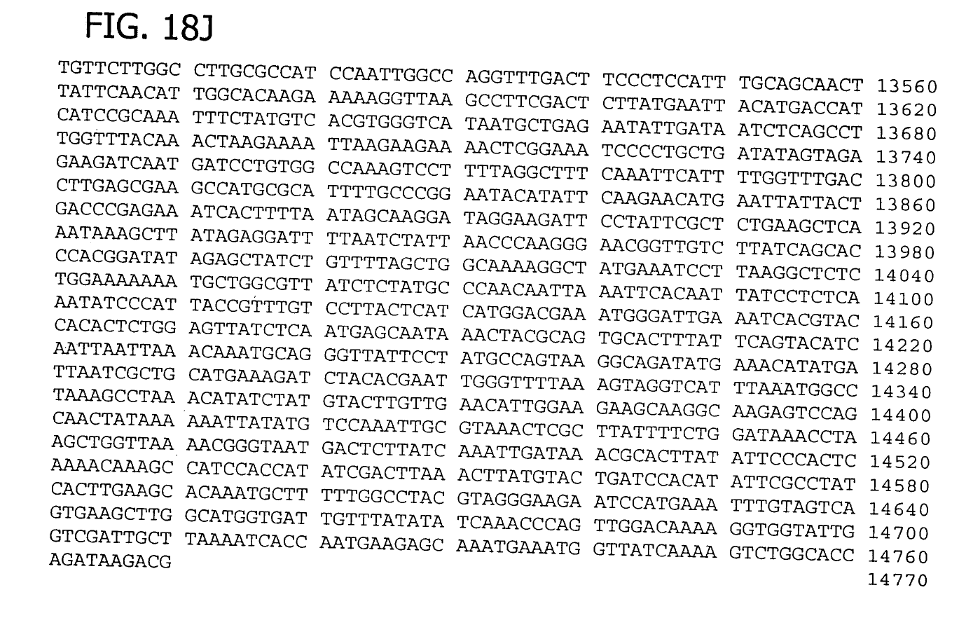

The invention further provides a rtxA gene of Legionella pneumophila. In particular, Example 1 describes the identification, isolation and characterization of the rtxA gene of L. pneumophila strain AA100. Southern hybridization analysis using this gene showed that rtxA genes are conserved in, and specific to, L. pneumophila (see Example 1). The sequence of the coding region of the rtxA gene of strain AA100 and portions of the flanking regions is given in FIG. 18 and SEQ ID NO:1. In addition to the rtxA gene, a polynucleotide having at least 90% sequence identity with the rtxA gene as identified in SEQ. ID. NO. 1 and which encodes for a protein having the same biological function as the protein encoded by SEQ. ID. NO. 1 is also useful for this invention.

RtxA genes other than the rtxA gene of strain AA100 can be identified and isolated using methods well known in the art. For instance, probes based on the sequence of the rtxA gene of strain AA100 can be used to screen L. pneumophila genomic libraries for other rtxA genes.

Because, rtxA genes are conserved in, and specific for, L. pneumophila strains, these genes, and the mRNAs and proteins encoded by them, are excellent targets for the detection or quantitation of L. pneumophila strains. For instance, L. pneumophila may be detected or quantitated by polymerase chain reaction (PCR) using primers that will amplify at least a portion of a rtxA gene, by the use of DNA or RNA probes that will hybridize to a rtxA gene or mRNA, or by an immunoassay using antibodies that bind specifically to a rtxA protein.

To perform the PCR assay, DNA is obtained from a sample suspected of containing L. pneumophila. Also, mRNA may be obtained from such a sample and used to prepare cDNA which can be used as the template in the PCR. Methods of extracting total cellular DNA and RNA from cells and methods of preparing cDNAs are well known. However, PCR does not require highly purified DNA, and DNA released by boiling of cells can be used directly without any purification.

The PCR primers are nucleic acid molecules having sequences selected so that the molecules hybridize to one of the strands of a rtxA gene. Of course, at least two primers must be used (one hybridizing to each of the strands of the rtxA gene), but more than one pair of primers can be used if it is desired to amplify more than one portion of the rtxA gene. The specificity of the primers should be confirmed by Southern blotting. Next, the DNA is amplified by PCR. PCR methods, equipment, and reagents are well known and are available commercially.

Finally, the amplified DNA is detected or quantified. This can be accomplished in a number of ways as is known in the art. For instance, the reaction mixture can be electrophoresed on agarose gels, and the presence or absence of amplified DNA of the expected size(s) can be determined by staining the gels. Alternatively, a labeled nucleic acid molecule which hybridizes to the amplified DNA (a probe) can be used to allow for detection or quantitation of the amplified DNA. As another alternative, the primers can be labeled, or the nucleotides used during the PCR can be labeled, and the labels incorporated into the amplified DNA can be detected or quantified.

Legionella pneumophila can also be detected or quantitated by obtaining DNA or RNA from a sample suspected of containing L. pneumophila. This can be accomplished in the same manner as described above for PCR analysis. The DNA or RNA is contacted with a probe which is a nucleic acid molecule having a sequence selected so that the molecule hybridizes to a rtxA gene or to mRNA encoded by a rtxA gene. The probe is allowed to hybridize with the DNA or RNA obtained from the sample. To detect or quantitate the rtxA gene or mRNA, the probe is labeled. The probe should be as large as possible while retaining specificity.

The invention further comprises the nucleic acid molecules used as probes and primers in these techniques. These nucleic acids molecules have sequences selected so that the molecules hybridize to a rtxA gene or an mRNA encoded by a rtxA gene. These nucleic acid molecules could also include antisense RNA molecules and ribozymes. Methods of making nucleic acid molecules are, of course, well known in the art.

The probes or primers may be labeled to allow for detection or quantitation of L. pneumophila. Suitable labels and methods of attaching or incorporating them into nucleic acid molecules are well known. Suitable labels include radioactive labels (e.g., .sup.32 P), chemiluminescent labels, fluorescent labels (e.g., fluorescein, rhodamine), particulate labels (e.g., gold colloids), calorimetric labels (e.g., dyes), enzymes, and biotin.

As noted above, labeled nucleotides can also be used during PCR to generate an amplified DNA which is labeled. The nucleotides are preferably labeled with radioactive labels (e.g., .sup.32 P) by methods well known in the art.

The invention also provides a kit containing reagents useful for detecting or quantifying L. pneumophila. The kit comprises at least one container holding at least one nucleic acid molecule of the invention (a probe or primer). For PCR assays, the kit will comprise at least two primers, which may be in the same container or in separate containers. The probes or primers may be labeled. The kit may contain other reagents and equipment useful in performing the assay, including PCR reagents (e.g., polymerase, labeled or unlabeled nucleotides), reagents for extraction of DNA or RNA, buffers, salt solutions, containers, gels and membranes, etc.

L. pneumophila may also be detected or quantitated in an immunoassay using antibodies that bind specifically to a rtxA protein. RtxA proteins or peptides can be used to produce antibodies useful in the immunoassay by methods well known in the art. A rtxA gene or fragment thereof may be used to produce a rtxA protein or peptide. Peptides can also be prepared by solid phase synthetic methods. Single chain or other engineered antibodies or fragments of antibodies containing an antibody combining site can be also used.

To perform the immunoassay, a sample suspected of containing L. pneumophila is contacted with the antibody under conditions so that the antibody can bind to a rtxA protein, if present. Standard immunoassay formats can be used. The antibody bound to the rtxA protein may be labeled to allow for detection or quantitation of the rtxA protein, or a labeled moiety that binds to the antibody (e.g., a secondary antibody directed to the first antibody or protein A) may be used. Suitable labels and methods of attaching them to antibodies and other proteins and compounds are well known. Suitable labels include radioactive labels (e.g., .sup.125 I), fluorescent labels (e.g., fluorescein, rhodamine), chemiluminescent labels, particulate labels (e.g., gold colloids), colorimetric labels (e.g., dyes), enzymes, and biotin.

Reagents useful for detecting or quantifying L. pneumophila by immunoassay may also be supplied as a kit. The kit will comprise at least one container holding an antibody that binds specifically to a rtxA protein. The kit may contain other reagents and equipment useful in performing immunoassays, including buffers, containers (e.g., test tubes, culture plates), substrates for enzyme labels, labeled streptavidin or avidin to bind to a biotin label, etc.

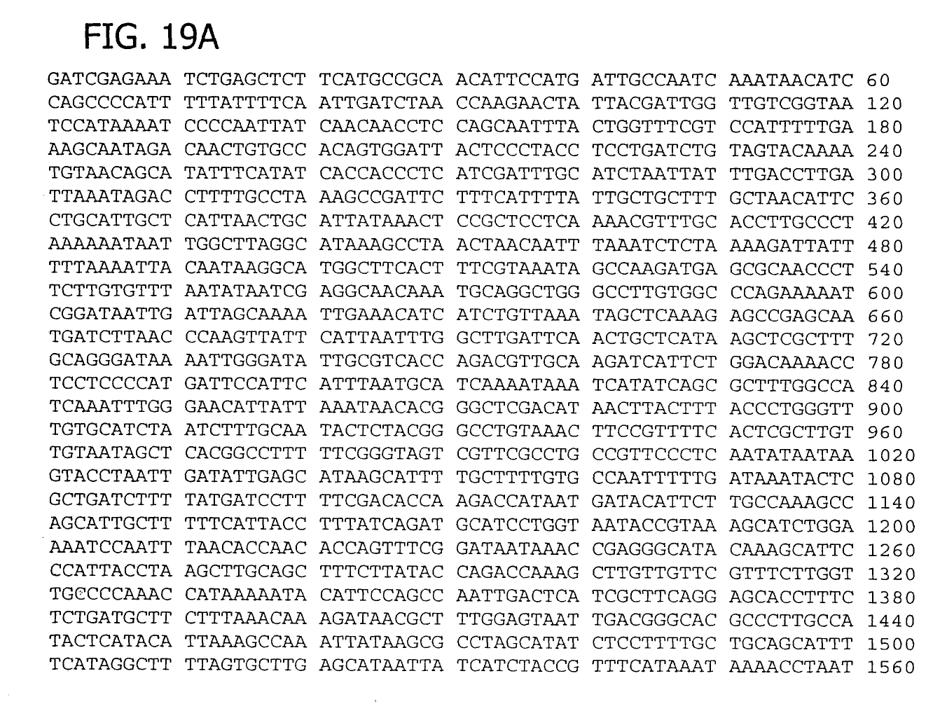

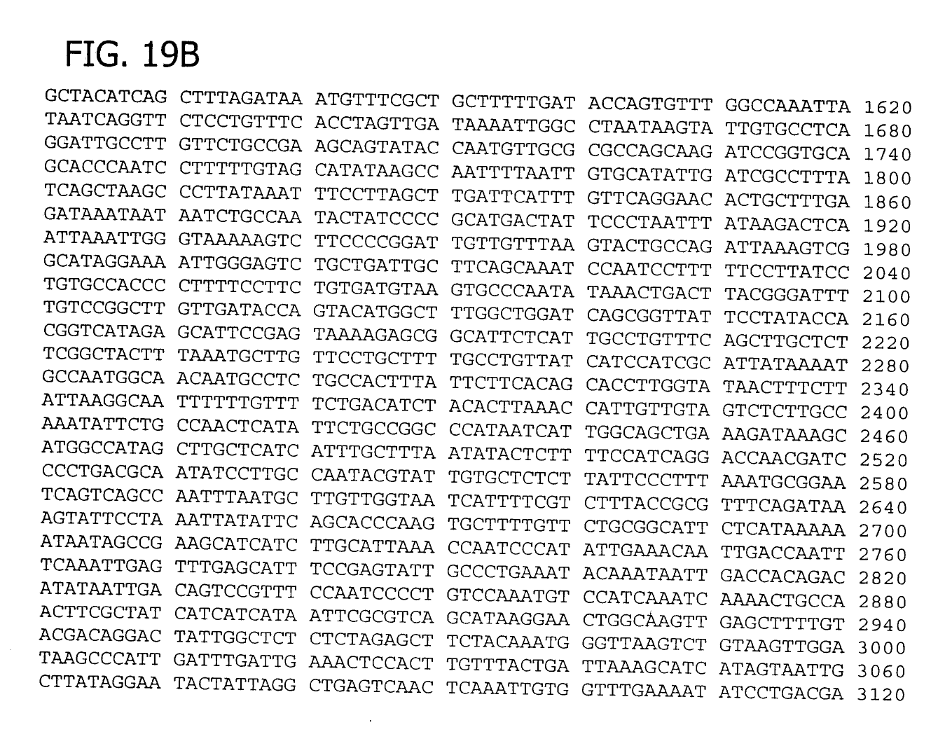

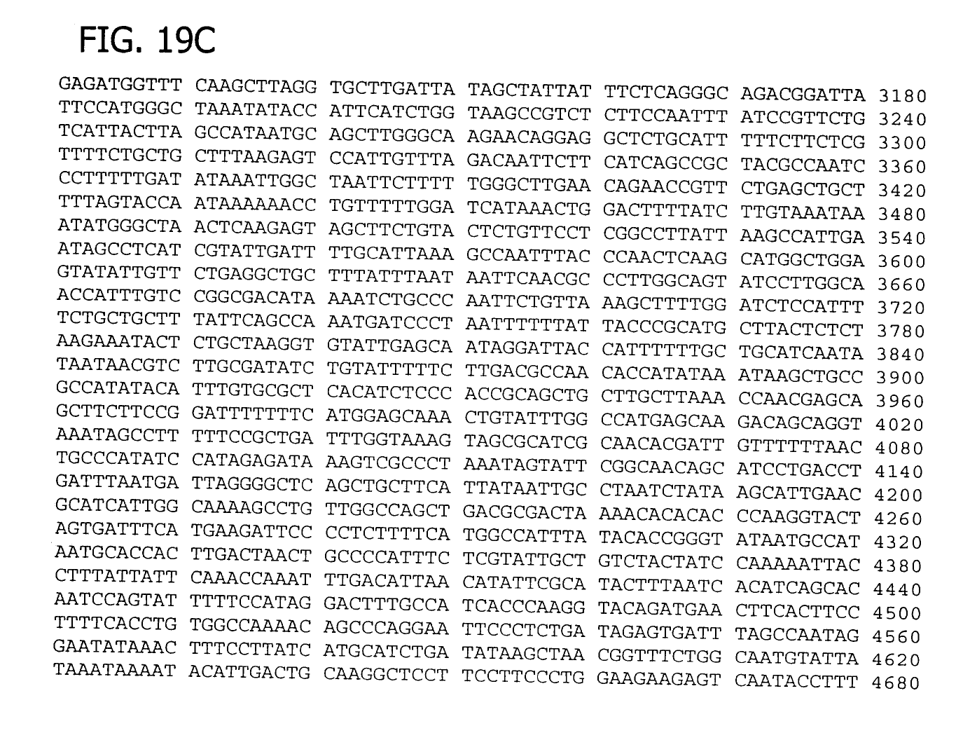

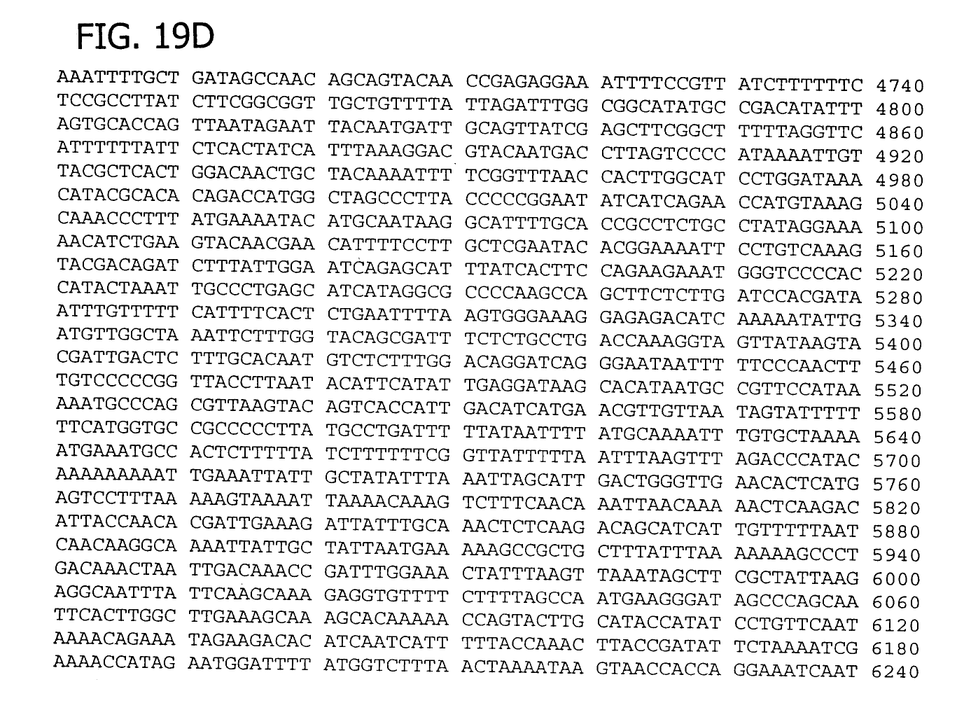

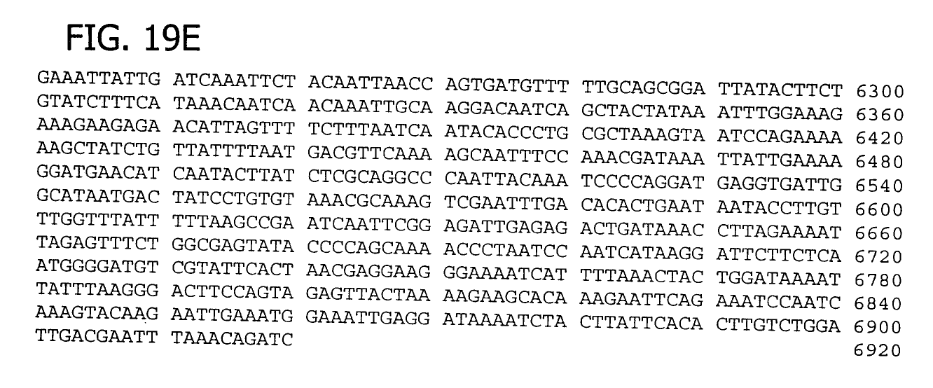

The invention further provides an enhC gene of L. pneumophila. In particular, Example 1 describes the identification, isolation and characterization of the enhC gene of L. pneumophila strain AA100. Southern hybridization analysis using this gene showed that enhC genes are found almost exclusively in L. pneumophila strains. The sequence of the coding region of the enhC gene of strain AA100 and portions of the flanking regions is given in FIG. 19 and SEQ ID NO. 2. In addition to the enhC gene, a polynucleotide having at least 90% sequence identity with the enhC gene as identified in SEQ. ID. NO. 2 and which encodes for a protein having the same biological function as the protein encoded by SEQ. ID. NO. 2 is also useful for this invention. enhC genes other than the enhC gene of strain AA100 can be identified and isolated using methods well known in the art. For instance, probes based on the sequence of the enhC gene of strain AA100 can be used to screen L. pneumophila genomic libraries for other enhC genes.

Because enhC genes are found almost exclusively in L. pneumophila strains, enhC genes, and the mRNAs and proteins encoded by them, are good targets for the detection or quantitation of L. pneumophila strains. For instance, L. pneumophila may be detected or quantitated by polymerase chain reaction (PCR) using primers that will amplify at least a portion of an enhC gene, by the use of DNA or RNA probes that will hybridize to an enhC gene or mRNA, or by an immunoassay using antibodies that bind specifically to a enhC protein. In particular, if high-stringency conditions are used for hybridizing probes to an enhC gene, mRNA or PCR-amplified DNA, the assays will differentiate L. pneumophila from other Legionella species. Also, the use of enhC-specific probes should also enhance the specificity of PCR assays as compared to current assays using primers directed to genes found in all Legionella species. These assays are performed as described above for the rtxA gene, mRNA and protein. Also, reagents for performing these assays are the same as described above, except that they are targeted to an enhC gene, an mRNA encoded by an enhC gene, or an enhC protein. The reagents may be supplied in kit form as described above.

Samples that may be used in the methods of the invention include water samples and clinical samples. Water samples may be taken from any source, including plumbing fixtures, evaporative condensers, cooling towers and supplies of potable water (e.g.,lakes and rivers). Clinical samples include sputum, throat swabs, blood, urine, cerebrospinal fluid, skin, biopsies, saliva, synovial fluid, bronchial lavages, or other tissue or fluid samples from humans or other animals.

Many of the procedures described above utilize hybridization of nucleic acids. Hybridization conditions and guidelines for selecting high- and low-stringency conditions are well known in the art. See, e.g., Sambrook et al., Molecular Cloning: A Laboratory Manual (2nd ed. 1989). Suitable hybridization conditions can be readily determined and optimized using only ordinary skill in the art.

The above-described assays targeted to a rtxA gene, an mRNA encoded by a rtxA gene, or a rtxA protein, or to an enhC gene, an mRNA encoded by an enhC gene, or an enhC protein may be used alone or in combination with each other or with other tests to detect or quantitate L. pneumophila. For instance, a PCR assay could utilize primers directed to a gene common to all Legionella in combination with primers directed to the rtxA gene which is specific to L. pneumophila.

The following example is intended to illustrate but not to limit the present invention. In light of the detailed description of the invention and the example presented below, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions below include some conclusions about the way the invention may function, the inventor does not intend to be bound by those conclusions and functions, but puts them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the claims.

### EXAMPLES

### Example 1

Bacterial Strains, Plasmids and Growth Conditions:

The strains used for these experiments are described in Table 1 below.

TABLE 1

| STRAIN/PLASMID | CHARACTERISTICS | SOURCE/REFERENCE |
|---|---|---|
| Strain: | | |
| E. Coli | | |
| XL1-Blue | recA1 endA1 gyrA96 thi-1 | Stratagene Cloning Systems, La |
| | hsdR17 supE44 relA1 lac [F′ | Jolla, Ca |
| | proAB lacI^{q}ZΔM15 Tn10 (Tet^{r})] | |
| χ2819 | F-lacY1 glnV44 galK2 galT22 | R. Curtiss, III |
| | λ(cI857 b2 redβ3 S7) recA56 | |
| | ΔthyA57 metB1 hsdR2 | |
| L. pneumophila | | |
| AA100 | 130b naturally arising Sm^{r} | N. C. Engleberg, et al. |
| | strain | |
| C7 | AA100 displays enhanced entry | Current study |
| | (Enh1) | |
| C8 | AA100 Enh1 | Current study |
| C1 | AA100 Enh2 | Current study |
| C2 | AA100 Enh2 | Current study |
| C9 | AA100 Enh2 | Current study |
| C10 | AA100 Enh2 | Current study |
| C3 | AA100 Enh3 | Current study |
| C11 | AA100 Enh3 | Current study |
| Plasmid: | | |
| pYUB289 | Km^{r} Ap^{r} p15Aori cos | V. Balasubramanian |
| 1A3 | Enh1 Km^{r} Ap^{r} p15Aori cos | Current study |
| 2A4 | Enh2 Km^{r} Ap^{r} p15Aori cos | Current study |
| 2A6 | Enh2 Km^{r} Ap^{r} p15Aori cos | Current study |
| pKV32 | Cm^{r} Ap^{r} | K. Visick, et al. |
| pJDC19 | enh2 (C3) Km^{r}Ap^{r} p15Aori cos | Current study |
| pJDC20 | enh1 (AA100) Km^{r} Ap^{r} p15Aori | Current study |
| | cos | |
| pJDC23 | enh1 (C3) Km^{r} Ap^{r} p15Aori cos | Current study |
| pJDC24 | enh2 (AA100) Km^{r} Ap^{r} p15Aori | Current study |
| | cos | |

The L. pneumophila AA100 strain has been shown to be virulent in both in vitro and in vivo models of infection. See Moffat, et al., “Effects of an isogenic Zn-metalloprotease-deficient mutant of Legionella pneumophila in a guinea-pig model,” Molec. Microbiol. 12:693-705 (1994). The L. pneumophila AA100 strain was passaged no more than twice in the laboratory before use in these studies to prevent loss of virulence. AA100 was grown on BCYE agar for three (3) days at 37° C. in 5% CO₂. The Escherichia coli K-12 strain XL1-Blue was grown in Lennox broth (LB, Difco Laboratories) at 37° C. When necessary, kanamycin and/or chloramphenicol (Sigma) were added at a concentration of 25 μg/ml to bacterial growth media.

Cell Culture:

HEp-2 cells (ATCC CCL23), established from a human epidermoid carcinoma, were grown in RPMI 1640 plus 5% heat inactivated fetal calf serum (GIBCO). THP-1 cells (ATCC TIB202), a human monocytic cell line, were grown in RPMI 1640 plus 10% heat inactivated fetal calf serum.

Entry and Adherence Assays:

HEp-2 cells were seeded in 24 well tissue culture dishes (Falcon) at a concentration of 2.5×10⁵ cells/well and allowed to adhere overnight at 37° C. The bacteria to be assayed were suspended and diluted in the same medium as the cells were, then washed with phosphate buffered saline (PBS) and incubated in the appropriate culture medium plus 100 μg/ml gentamicin for 2 hours. After antibiotic treatment the cells were washed with PBS, then with water and lysed by incubation for 10 minutes in one ml of water followed by vigorous pipetting. In the case of THP-1 cells, the bacteria were allowed to interact with cells for 30 minutes and the assays were caried out in suspension. This requires that the cells be pelleted by centrifugation at 100×g for one minute before each change of solution. After lysis the number of intracellular bacteria was determined by plating for colony forming units (cfu) on BCYE (L. pneumophila) or LB agar (E. Coli). Entry levels were determined by calculating the percentage of inoculum that becamegentamicin resistant over the course of the assay (i.e. percent entry=100×(cfu gentamicin resistant/cfu inoculum)). In order to correct for variation in levels of uptake between experiments, entry is reported relative to AA100 (i.e. relative entry=percent entry test strain/% entry AA100). Adhesion was tested in a similar manner to that for entry, except that bacteria were added to the cells, mixed and immediately washed three times to remove non-adherent bacteria prior to lysis.

Chemical and Transposon Mutagenesis:

AA100 was mutagenized at a concentration of 1×10⁸ cfu/ml with 30 μg/ml ethylmethane sulfonate (EMS) for 1 hour at 37° C. in a minimal A buffer. This method of EMS treatment resulted in a level of 1.25% survival of L. pneumophila. The pool of mutagenized bacteria was then washed twice in minimal A buffer and grown overnight on BCYE agar. The pool was suspended in RPMI 1640 and aliquots (106 cfu)were used to isolate enhanced entry mutants.

Cosmids carrying L. pneumophila genomic fragments were mutagenized with a chloramphenicol resistant mini-Tn10 transposon carried on plasmid pKV32. See Alexeyev, et al., “Mini-Tn10 trasposon derivatives for insertion mutagenesis and gene delivery into the chromosome of Gram-negative bacteria,” Gene 160:9-62 (1995). The plasmid pKV32 has an R6K γ-origin of replication, which prohibits its replication in strains that do not carry the π protein in trans. Mutagenesis of cosmids 1A3, 2A4, and 2A6 with this trasposon was carried out by transformation of XL1-Blue strain containing each of these cosmids with pKV32. The resulting transformants were then allowed to recover in SOC medium with 2 mM IPTG for one hour at 37° C. to induce trasposition. The culture was then incubated for an additional four (4) hours in the presence of kanamycin and IPTG. Aliquots were plated on LB agar plates with kanamycin and chloramphenicol. The 100-200 colonies that arose from transposition events were pooled, plasmid DNA isolated by alkaline lysis and transformed in to XL1-Blue. See Sambrook, et al., Molecular cloning: a laboratory manual, 2 ed., Cold Spring Harbor Laboratory, Cold Spring Harbor, N.Y. (1989). This procedure allows isolation of specifically those transposition events into the plasmid. Individual colonies were then screened for the presence of unique insertions into the plasmid by restriction analysis. Unique transposon insertions were then electroporated into AA100 and tested for the ability to confer enhanced entry.

Selection for Enhanced Entry Mutants:

Mutants that displayed enhanced entry were isolated through the use of a modified HEp-2 cell entry assay. This selective entry assay was accomplished by growing EMS mutagenized L. pneumophilla strain AA100 on BCYE agar at 37° C. for five (5) days and then pooling the resulting colonies in RPMI. A standard entry assay into HEp-2 cells was carried out with this suspension as described above except that the bacteria were only allowed to interact with the HEp-2 cells for five (5) minutes. The bacteria that entered during this assay were grown on BCYE agar for three (3) days and compared to AA100 in a standard entry assay. Those clones that displayed greater than two-fold increase in entry over AA100 were considered to have an Enh phenotype.

Library Construction and Screening for Dominant Muations:

Contiguous and non-contiguous L. pneumophila strain AA100 and C3 total genomic DNA libraries were constructed in the cosmid vector pYUB289. The cosmid pYUB289 was constructed by first producing a PacI cassette in SuperCos I (Stratagene) as described previously for cosmid pYUB328. See Balasubramanian, et al., “Allelic exchange in Mycobacterium tuberculosis with long linear recombination substrates,” J. Bacteriol. 178:273-279 (1996). The resulting cassette and adjacent cos site were moved to pACYC177 by digestion with NheI and AatII, isolation of the appropriate DNA fragments and ligation. The resulting cosmid carries a single cos site, the PacI cassette, kanamycin resistance, ampicillin resistance and the low copy-number origin of replication p15A. In order to construct the Legionella genomic libraries, total genomic DNA was isolated as described for E. coli, digested partially with Sau3AI to produce fragments of approximately 20 Kbp in length for non-contiguous libraries and 50 Kbp for contiguous libraries. See Skaliy, et al., “Survival of Legionnaires' disease bacterium in water,” Ann. Intern. Med. 90:662-663 (1979). For non-contiguous libraries, these fragments were ligated to BamHI cut and dephosphorylated pYUB289. For contiguous libraries these fragments were dephosphorylated and ligated to BamHI cut pYUB289. The resulting ligations were in vitro packaged with Gigapack II Gold (Stratagene) packaging mix and used to infect χ2819 for in vivo packaging. Over 10,000 kanamycin resistant χ2819 colonies were pooled for in vivo packaging, producing a lysate that had a titer of grater than 10⁹ cosmid containing phage/ml. This lysate was used to infect XL1-Blue and plated on LB agar plates containing kanamycin such that approximately 20,000 colonies were produced on ten plates. The resulting colonies were pooled in two separate pools of approximately 10,000 colonies each and plasmid prepared from them.

In order to screen for dominant mutations, the two resulting plasmid preparations from the non-contiguous C3 library were electroporated independently into AA100 and dilutions plated on BCYE agar with kanamycin. Approximately 10,000 kanamycin resistant AA100 colonies from eahc transformation were pooled and clones that conferred the enhance entry phenotype isolated in the same manner as the original isolation of EMS mutagenized enhanced entry mutants. Individual colonies were isolated following the selective entry assay and their ability to enter HEp-2 cells compared to wild-type AA100. Those clones that entered HEp-2 cells at two-fold higher frequencies than AA100 were considered to confer the Enh phenotype. For each of the transposon insertions, the ability to confer the Enh phenotype was tested in a similar manner except that the purified plasmids carrying insertions were directly transformed into AA100 and tested for the Enh phenotype.

To ensure that the cosmid carried by each clone was responsible for the enhanced entry rather than the acquisition of a spontaneous mutation in the host bacteria, each cosmid that conferred the Enh phenotype was transferred from AA100 into XL1-Blue, purified, re-transformed into AA100 and re-tested in the entry assay. Cosmids were transferred into E. coli by the technique of direct electroporation.

Isolation and Subcloning of Continuous Enh Loci:

In order to demonstrate that the Enh phenotype was due to the loci indicated by transposon mutagenesis and not the result of scrambled genes, we identified and subcloned each of the loci involved from the contiguous genomic libraries of C3 and AA100. A 609 bp region beginning 33 bp upstream of the putative translational start for rtxA was used as a probe in colony hybridizations to isolate the contiguous cosmid clones that contain the enh1 locus. A 438 bp region beginning 590 bp upstream of the putative translational stop for enhC was used as a probe in colony hybridizations to isolate the contiguous cosmid clones that contain the enh2 locus. Restriction and Southern analysis confirmed that the enh1 and enh2 loci on these cosmids were on a 5265 bp EcoRI and 5265 bp EcoRI and 5263 bp SwaI fragment, respectively, that was the same size as in total C3 and AA100 chromosomal DNA digests. One of each of the cosmids isolated in this manner containing enh1 and enh2 was digested with either EcoRI or SwaI, respectively, and the appropriate size fragment was purified and ligated into EcoRI or ScaI digested pYUB289. The resulting plasmids, PJDC19, pJDC20, pJDC23, and pJDC24 are listed in Table 1. The presence of the complete contiguous loci on each of these subclones was confirmed by restriction and sequence analysis.

DNA Manipulations and Direct Electroporation:

DNA manipulations were carried out as described previously. Plasmid DNA was prepared from Legionella pneumophila and E. coli by alkaline lysis. PCR reactions were carried out using the protocols recommended by the maker of the Taq (Perkin Elmer) polymerase used.

Direct electroporation proved to be a rapid and efficient method for transferring plasmids from Legionella pneumophila to E. coli. For this technique, colonies containing the plasmid of interest were suspended in 1 ml of sterile distilled water (SDW) at 4° C. in an eppendorf tube, washed twice with SDW and mixed 1:1 with freshly thawed XL1-Blue electroporation competent cells prepared and frozen as described previously. See Dower, et al., “High efficiency transformation of E. coli by high voltage electroporation,” Nucleic Acids Res. 16(13):6127-6145 (1988). Electroporation was then carried out in the same manner as for E. Coli and dilutions plated on LB agar with kanamycin.

Southern Analysis and Colony Hybridization:

Probes were labeled by nick translation or PCR with digoxigenin using the methods suggested by the manufacturer of the Genius System (Boehringer-Mannheim). Membranes were prepared by the suggested methods for both colony hybridization and Southern analysis. Hybridization and washes were carried out at high stringency.

DNA Sequence Analysis:

DNA sequence analysis was carried out initially using a forward primer from the transposon, TrnF (CCACTAGTTCTAGAGCGGCC)(SEQ ID. 3). The sequence was continued by primer walking directly on the cosmid of interest. All regions were sequenced completely in both directions using BigDye Terminator (PE Applied Biosystems) cycle sequencing and subsequent analysis on an ABI 310 automated sequencing apparatus (PE Applied Biosystems). Sequence analysis and assembly was carried out using Gene Construction Kit 2 (Textco, Inc.) and comparison with known sequences using BLAST. See Altschul, et al., “Gapped BLAST and PSI-BLAST: a new generation of protein database search programs,” Nuc. Acids Res. 25:3389-3402 (1997). Analysis of putative signal sequences was done using SignalP, potential protein profiles using ProfileScan and secondary structure and multiple sequence alignments using Lasergene (DNASTAR, Inc.) software. See Nielsen, et al., “Identification of prokaryotic and eukaryotic signal peptides and predictions of their cleavage sites,” Prot. Engineer 10:1-6 (1997); Luthy, et al., “Improving the sensitivity of the sequence profile method,” Prot. Csi. 3:139-146 (1994).

Nucleotide Sequence Accession Numbers and Statistical Analysis:

The sequences of the enh1 and enh2 loci have been assigned the GenBank accession numbers AF057703 and AF057704, respectively.

All in vitro experiments were carried out in triplicate and repeated at least twice. Significance of the results was analyzed using ANOVA. Values of p<0.05 were considered significant.

Results

Isolation of L. pneumophila Enhanced Entry Mutants:

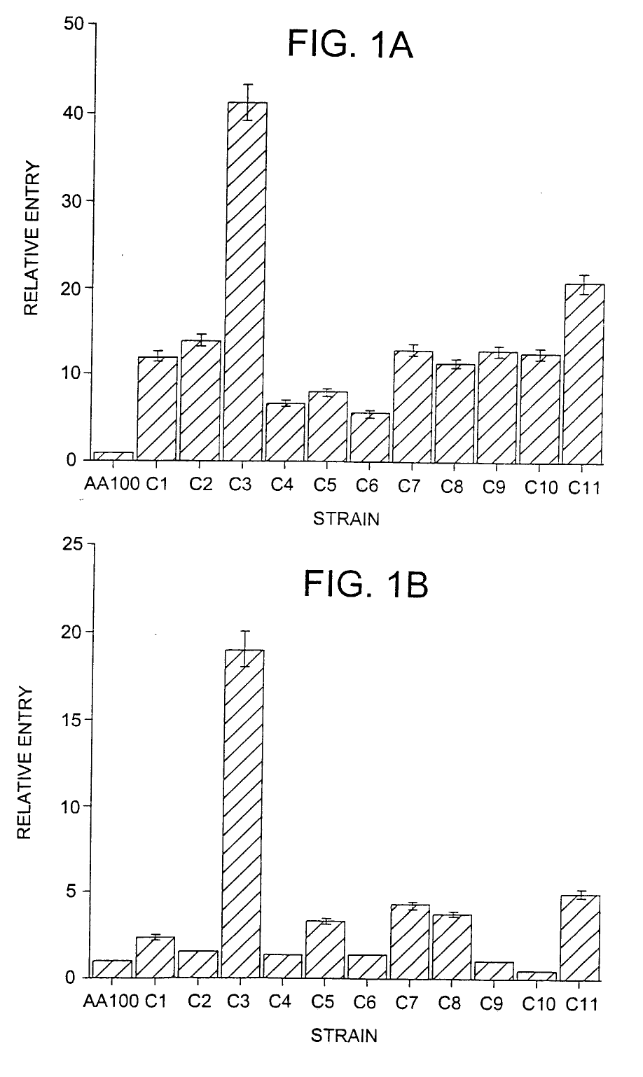

EMS mutagenesis was used to produce a pool of mutagenized L. Pneumophila that is enriched for mutants that have acquired an enhanced ability to enter cells when grown on BCYE agar. Enh entry mutants were isolated using selective entry assays from two independent EMS mutagenized AA100 pools of approximately 106 cfu. Twenty clones isolated from each selection were screened individually for their ability to enter HEp-2 cells relative to wild-type AA100. Results from a representative assay are shown in FIG. 1A. From the 40 clones screened in HEp-2 cells, 22 clones or approximately 55% displayed the Enh entry phenotype. Thus, the selection procedure was relatively successful for the isolation of Enh mutants. Eleven of these clones were also assayed for their ability to enter THP-1 cells (FIG. 1B). These clones were classed into three phenotypic groups: Enh1(C7 and C8) that enter both HEp-2 and THP-1 cells at greater than two-fold higher levels than wild-type but not THP-1 cells; and Enh3 (C3 and C11) that enter both HEp-2 and THP-1 cells at greater than five-fold higher levels than wild-type.

Isolation of Cosmids that Confer the Enh Phenotype to Wild-Type L. pneumophila:

The Enh mutant C3 was chosen for further anaylsis due to its high level of enhanced entry into both HEp-2 and THP-1 cells. We first compared the phenotypic characteristics of the C3 mutant to wild-type L. pneumophila. We characterized the C3 Enh mutant in terms of intracellular replication rate, growth rate in laboratory media, the presence of pili, the presence of flagella, motility, colony morphology and ultrastructural morphology. None of these phenotypic characteristics were significantly different from wild-type L. pneumophila. The only apparent differences between the C3 mutant and wild-type L. pneumophila were in its adherence and entry phenotype.

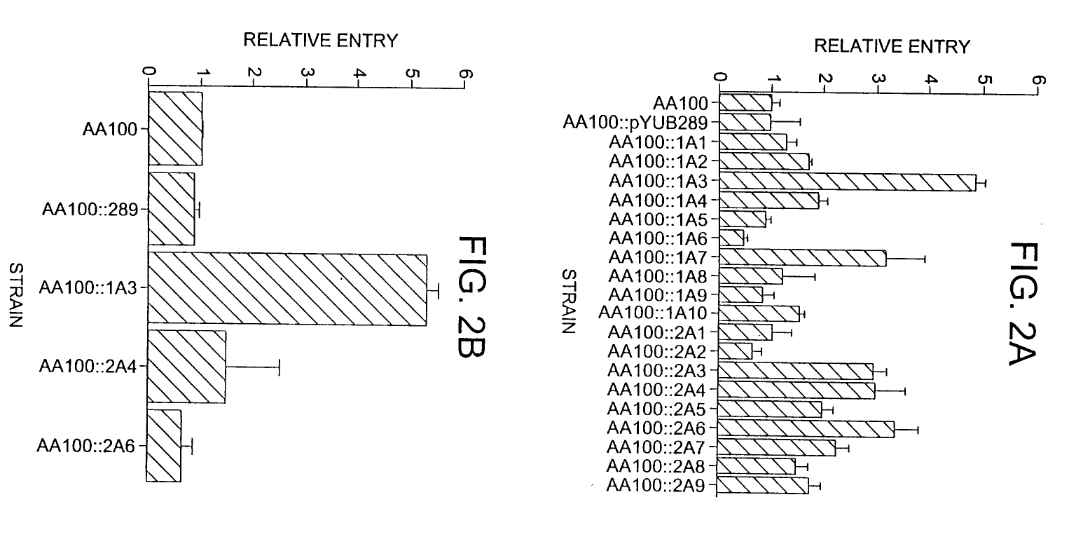

A cosmid library of C3 total genomic DNA was constructed and transferred into wild-type AA100 to allow isolation of the genes responsible for the C3 Enh phenotype. Due to the possibility that the C3 Enh phenotype was due to multiple mutations, we utilized a non-contiguous genomic library for these experiments. A low copy-number cosmid vector was chosen for these studies to reduce the possibility of copy-number effects, such as toxicity of L. pneumophila gene products in E.coli, on the comprehensiveness of the library. Recombinant cosmids that conferred the Enh phenotype were isolated using selective entry assays. A total of 19 cosmids were isolated from two independent pools of 20,000 AA100 transformants with the C3 genomic library. The ability of each of these recombinant clones to enter HEp-2 cells was compared to wild-type in standard entry assays (FIG. 2A). Seven cosmid containing clones consistently displayed the Enh phenotype, two from the first pool (containing 1A3 and 1A7) and five from the second pool (containing 2A3, 2A4, 2A6, 2A7 and 2A9). A restriction map of each of these cosmids was constructed using BamHI. All of these cosmids had unique restriction maps excluding 2A4 and 2A6, which were identical. Cosmid 1A3 consistently displayed the highest relative entry of those tested. On the basis of these data, cosmids 1A3, 2A4 and 2A6 were chosen for further analysis.

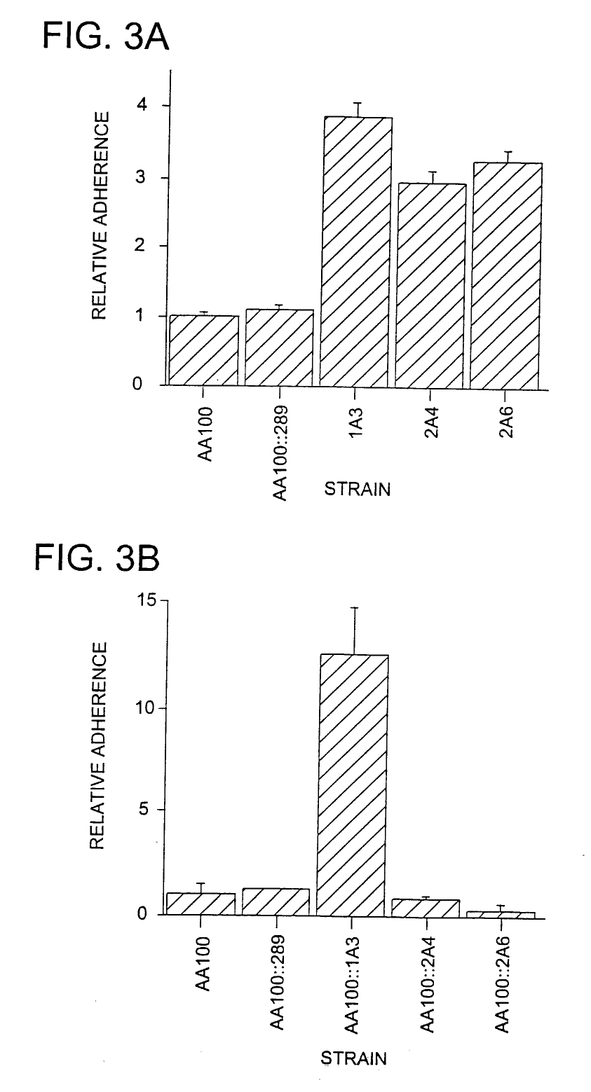

Cosmids 1A3, 2A4 and 2A6 were purified in E. Coli and then re-transformed into AA100 to determine whether the Enh phenotype conferred by these cosmids was due to the plasmid or a spontaneous mutation in the host bacterium. Transformations from AA100 to E. coli were carried out by direct electroporation. The ability of AA100 carrying the purified cosmids relative to wild-type L. pneumophila to enter HEp-2 cells was then determined. Entry of these transformations was comparable to that obtained with the original clones carrying the cosmids. These results indicate that the observed phenotype can be attributed to activities present on the cosmid rather than in the host bacterium. The entry phenotypes of 2A4 and 2A6 into monocytes were the same as wild-type L. pneumophila (FIG. 2B). Only cosmid 1A3 conferred the Enh phenotype into both monocytes and epithlial cells, similar to the original C3 Enh mutant. We assayed the adherence of these clones to HEp-2 cells and THP-1 cells to determine whether adherence was affected as well as entry. We found a direct correlation between the entry and adherence phenotype of all of the clones isolated (FIG. 3). These data suggest that adherence and entry are intimately linked in L. pneumophila.

Identification of Enhanced Entry (Enh) Loci:

Detailed physical maps of cosmids 1A3, 2A4 and 2A6 were constructed using restriction enzymes BamHI, NehI and XhoI. Cosmids 2A4 and 2A6 have identical physical maps with these three enzymes. However, cosmid 1A3 has no apparent regions of overlap with the cosmids 2A4 and 2A6. Southern analysis was carried out with AA100 chromosomal DNA and purified 1A3, 2A4 and 2A6 plasmid DNA digested with BamHI using 1A3 and 2A6 separately as probes. No overlapping regions were identified by Southern analysis for 1A3 with the 2A4 and 2A6 cosmids. However, 2A6 hybridized with all restriction fragments of the cosmid 2A4, indicating that these cosmids are indentical. Thus, only cosmids 1A3 and 2A6 were analyzed further.

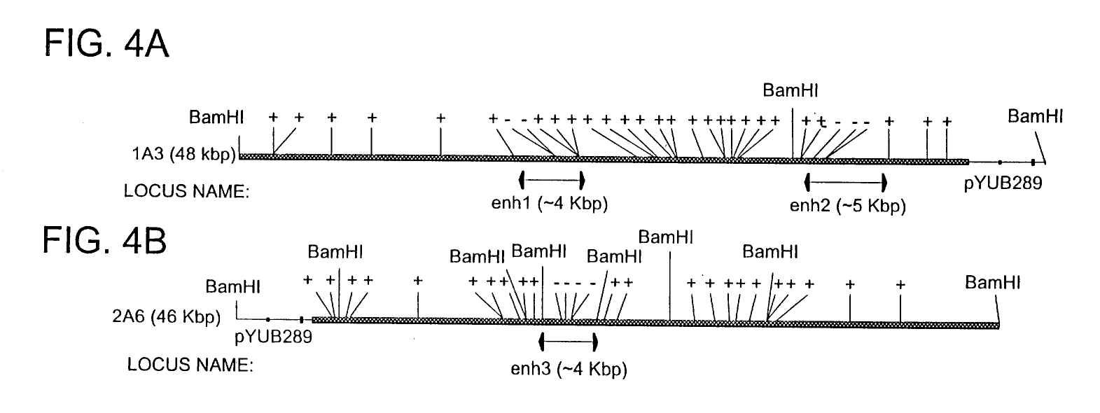

Transposon mutagenesis of cosmids 1A3 and 2A6 was carried out in XL1 Blue using mini-Tn10. Greater than 100 transposon insertions in each cosmid were isolated and partially mapped with restriction endonucleases to determine the location of each transposon insertion. Approximately 30 unique transposon insertion per cosmid family were tested for their ability to confer the Enh phenotype of the original cosmid in HEp-2. The location and phenotype of each transposon insertion is shown in FIG. 4. Two different loci, designated enh1(˜4 Kbp) and enh2 (˜5 Kbp), on cosmid 1A3 were involved in the ability of this cosmid to confer the Enh phenotype (FIG. 4). In the case of cosmid 2A6, however, only one locus, designated enh3 (˜4 Kbp), was required (FIG. 4). In order to confirm that each locus separate from adjoining fragments was sufficient to confer the Enh phenotype and this phenotype was due to the presence of contiguous chromosomal fragmetns rather than scrambled genes, we identified and subcloned these loci from out contiguous C3 and AA100 genomic libraries.

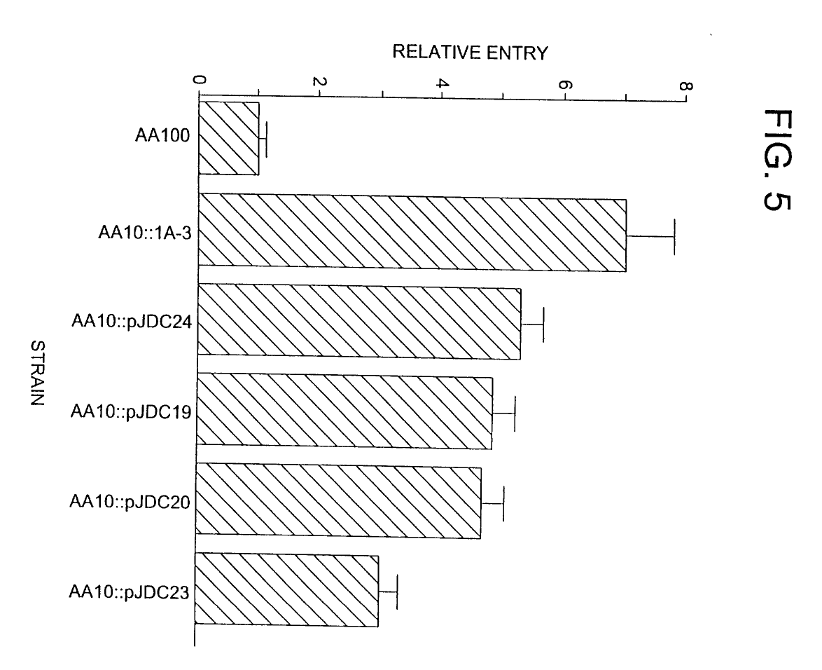

Colony hybridization was used to identify cosmids in these contiguous libraries that carry enh1 and enh2. Contiguous fragments of approximately 5 Kbp that contain each of these loci were subcloned into pYUB289 and the resulting plasmids pJDC19, pJDC20, pJDC23 and pJDC24 (Table 1) were tested in entry assays (FIG. 5). The contiguous fragmetns from both C3 and AA100 conferred the Enh phenotype. However, the levels of enhanced entry conferred by enh1 and enh2 separately were not quite as high as the original cosmid 1A3 that contains both loci. This data suggests that the presence of both loci has a synergistic effect on entry. In addition, the fact that both loci from C3 and AA100 confer similar levels of enhanced entry suggests that the Enh phenotype conferred by enh1 and enh2 is due to gene copy-number effects. This conclusion is further supported by sequence analysis of these loci from C3 and AA100.

Sequence Analysis of the Enh1 Locus:

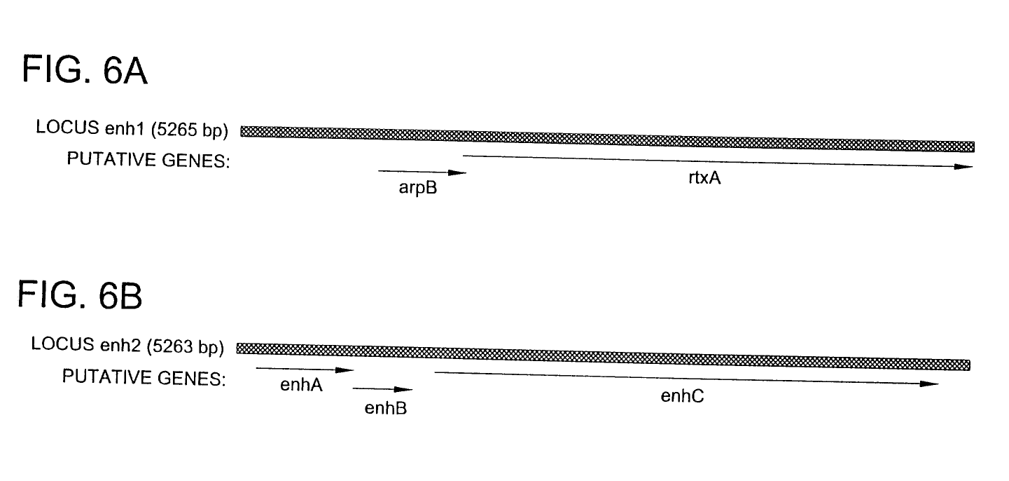

Initial sequence analysis was carried out from the mini-Tn10 insertions that resulted in an Enh negative phenotype on the cosmid 1A3. This strategy allowed rapid identification of the genes required to confer this phenotype. The two Enh negative transposon insertions in locus enh1 were within a gene that encodes a protein that has significant amino acid (a.a.) sequence identity to several leukotoxins or cytotoxins that are members of the repeats in structural toxin (RTX) family including frpC from Neisseria meningitidis (23% identity in 1106 a.a., hlaA from Pasteurella haemolytica (23% identity in 634 a.a.) and cyaA from Pordetella bronchiseptica and Bordetella pertussis (22% identity in 795 a.a.) This ORF was given the gene designiation rtxA based on this data. The complete sequence of the 5265 bp EcoRI fragment containing the enh1 locus was determined for both C3 and AA100 in this region. The region upstream of rtxA gene contains a second ORF (216 a.a.) that is transcribed in the same direction as the rtxA gene and overlaps it by 10 a.a. This ORF was found to have amino acid similarity to the asparagine-rich antigen (accession number 102334) from Plasmodium falciparum (25% identity in 166 a.a.) and the outer membrane usher protein (USEH) for CS6 fimbriae (accession number 1706159) from Escherichia coli (31% identity in 106 a.a.). This siilarity is primarily in asparagine-rich regions of the protein (FIG. 6). Upstream of this ORF there is a region of approximately 300 bp that does not appear to contain any large ORFs. A potential 10 sequence, AATATTG (SEQ ID. 4), was found 146 bp upstream and a potential 35 sequence, GTATTGTAAG (SEQ ID. 5), was found 178 bp upstream of the ATG for arpB. No other putative 10 and 35 sequences were found upstream of rtxA.

Sequence Analysis of the Enh2 Locus:

Sequence analysis of the enh2 locus was begun in the same manner as for enh1. Three mini-Tn10 insertions in the enh2 locus gave an Enh negative phenotype. Sequence analysis from these transposon insertions outward resulted int eh identification of an ORF with significant animo acid similarity to the sel-1 gene product from Caenorhabditis elegans (25% identity in 550 a.a.), the chitin synthase 4 gene product from Candida albicans (24% identity in 275 a.a.) and the skt5 gene product from Saccharomyces cerevisiae (25% identity in 361 a.a.). Sequence analysis was carried out on the complete 5263 bp enh2 locus for both C3 and AA100 encompassing this ORF and two other ORFs that appear to be transcribed in the same direction (FIG. 6). There were no differences between the nucleotide sequence of C3 and AA100 in this region. The two upstream ORFs transcribed in the same direction had no significant similarity to any sequence in the available protein and DNA sequence databases. Based upon the Enh phenotype of this locus, these ORFs were given the gene designations enhA, enhB and enhC. All three enh genes have an upstream region that closely resembles the E. coli consensus promoter sequence.

Characterization of the rtxA anad EnhABC Genes:

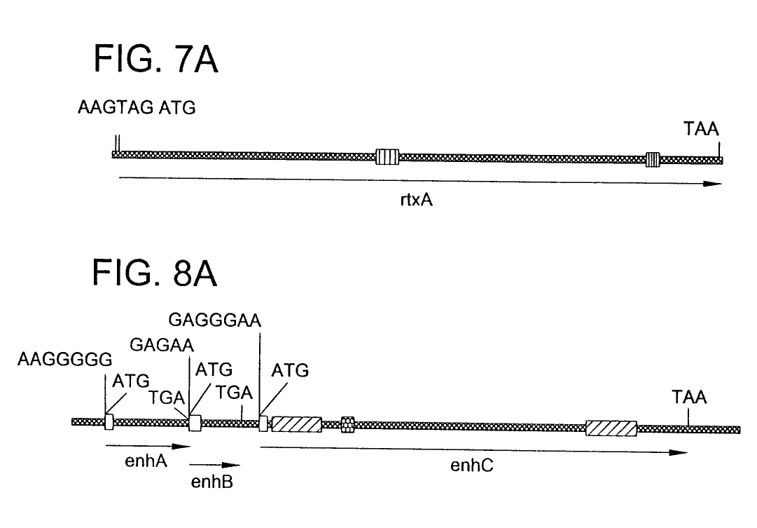

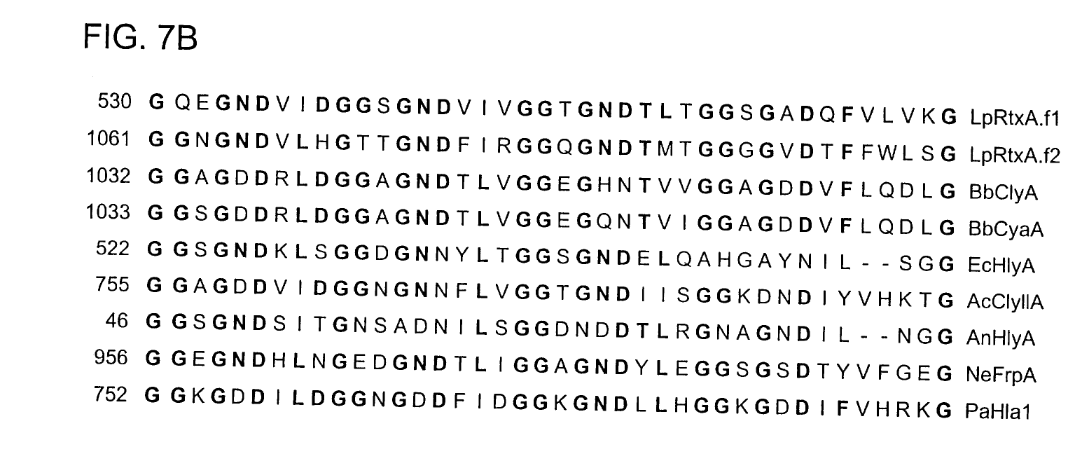

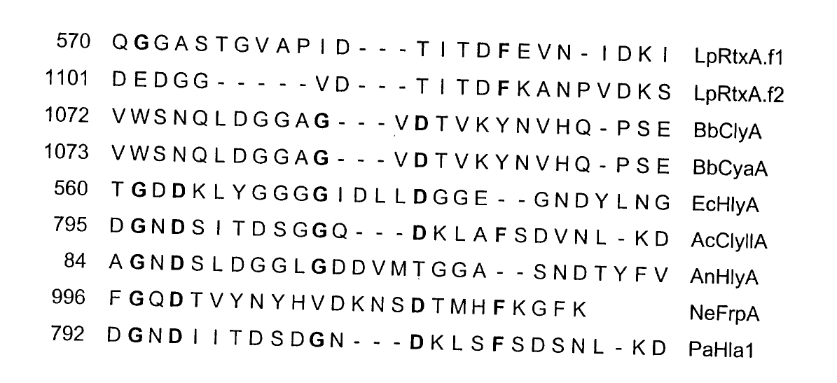

Analysis of the sequence of the rtxA gene revealed the presence of several distinctive characteristics (FIG. 7). Standard translational start (ATG) and stop (TAA) sequences were present, allowing the translation of a protein of 1208 a.a. However, a somewhat unusual ribosomal binding site (AAGTAG)(SEQ ID. 6) was found upstream of the translational start. Eight repeats of the RTX consensus sequence in two separate regions of the protein (at a.a. 519 and 1060) were found within the L. pneumophila rtxA gene (FIG. 7A). The first of these regions has five 9 a.a. RTX repeats, whereas the second region has only three. The 9 a.a. repeats were nearly identical for all copies present in the L. pneumophila rtxA gene. When the carboxy terminal regions of the RTX repeats were aligned, additional sequence similarity was observed in the 40 a.a. carboxy terminal of the repeats. An alignment of the two L. pneumophila RTX repeat regions with similar RTX regions from other species is shown in FIG. 7B. The positions of glycine, asparate and phenylalanine residues continue to be highly conserved in this region.

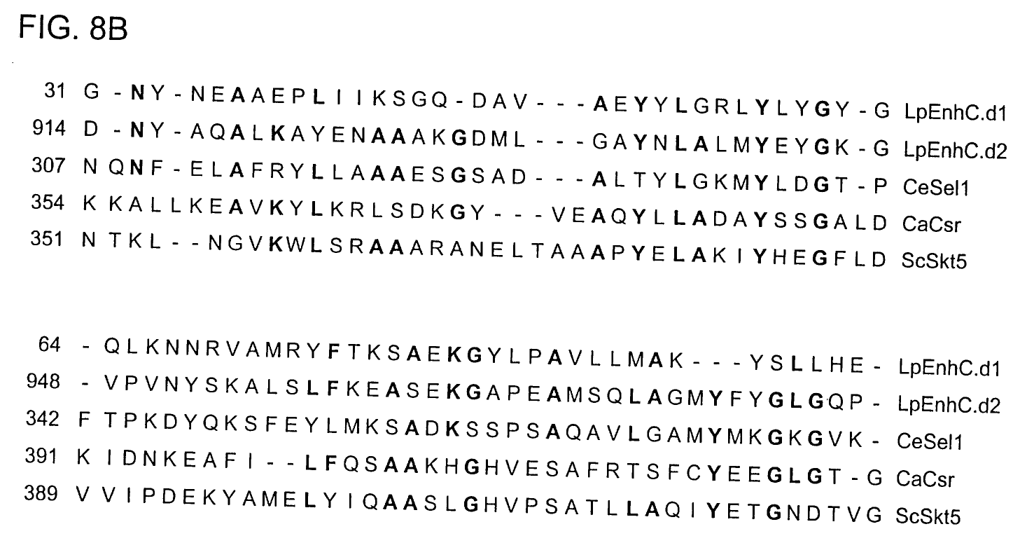

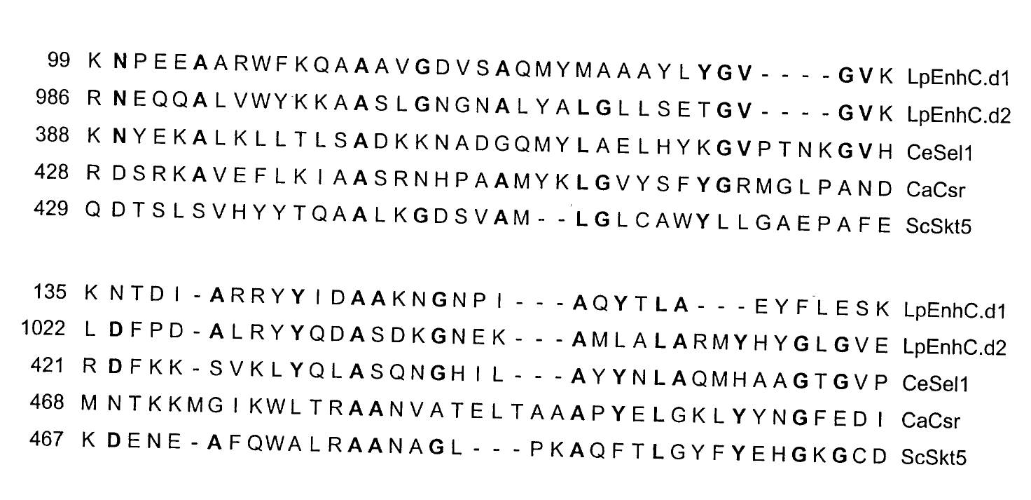

The enhA, enhB and enhC genes each have standard translational start, translational stop and ribisomal binding sites (FIG. 8A). The gene enhA would encode a protein of 240 a.a., enhB a protein of 142 a.a. and enhC a protein of 1200 a.a. In addition, all three genes encode proteins with a hydrophobic alpha-helical region in their amino terminus that has an amino acid sequence characteristic of standard secretory signal sequecnes in Gram-negative bacteria. The potential signal sequence cleavage sites for enhA, enhB, and enhC are between amino acids 19 and 20; 27 and 28; and 21 and 22, respectively.

The putative enhC gene product has several characteristics that are reminiscent of eukaryotic proteins. A region was found between amino acids 223 and 257 (FIG. 8A) that fits the profile for eukaryotic TPR regulatory proteins (nvalue=8.4). However, only one repeat region was found within enhC, whereas at least seven are usually found in other TPR proteins. See Harb, et al., “Heterogeniety in the attachment and uptake mechanisms of Legionnaires' disease bacterium, Legionella pneumophila, by protozoan hosts,” Environ. Microbiol. 64:126-132. The strongest amino acid sequence similarity between the protein encoded by enhC and known protein sequences was with the sel-1 gene product from C. elegans and the skt5 gene product from S. cerevisiae. There is a conserved domain of Sel-1 that has previously been observed to be similar to a region Skt5. See Grant, et al., “Structure, function, and expression of SEL-1, a negative regulator of LIN-12 and GLP-1 in C. elegans,” Development 124:637-644 (1997). This region of Sel-1 is the region of highest sequence similarity to enhC. However, instead of a single conserved region, enhC has two regions that have similarity to each other as well as to the conserved region of Sel-1 (FIG. 8). Alignment of the conserved regions of enhC with all of the proteins that display high similarity to enhC reveals the presence of this conserved region in the chitin synthase 4 gene from C. albicans as well (FIG. 8B). A characteristic of this region is conserved alanine repeats every 7-8 amino acids with highly conserved leucine, tyrosine and glycine residues. Prediction of the secondary structure of this conserved domain suggests a beta-sheet, alpha-helix, turn (proline at amine acid 62-63 in alignment), alpha-helix, beta-sheet structure. The alpha-helical domains are hydrophobic and represent potential transmembrane regions. The phenotypic effect of enhC on L. pneumophila entry and similarity to eukaryotic proteins, particularly Sel-1, suggests that enhC is a secreted protein that is involved in the interaction of L. pneumophila with host cells.

### Example 2

### Materials and Methods

Bacterial strains and growth conditions. The L. pneumophila strain used for these studies was the streptomycin-resistant variant of L. pneumophila serogroup 1 strain AA100. This strain has been shown to be virulent in both in vitro and in vivo models of infection and was passed no more than twice in the laboratory before use in these studies. AA100 was grown on BCYE agar for three days at 37° C. in 5% CO₂ as described previously. The Escherichia coli K-12 strain ψEC47, used for propagation of R6K ori plasmids, (XL1-Blue (Stratagene) lysogenized with Δpir) was grown in Lennox broth (LB, Difco Laboratories) at 37° C. When necessary, kanamycin (Sigma) was added at a concentration of 25 μg/ml, NaCl added at 5 mg/ml and sucrose at 50 mg/ml to bacterial growth media. Phenotypic characterization of strains for growth rate in laboratory media and sodium and osmotic sensitivity were carried out exactly as described previously See Byrne et al., “Expression of Legionella pneumophila virulence traits in response to growth conditions,” Infect Immun 66:3029-3034 (1998).

Cell culture. HEp-2 cells (ATCC CCL23), established from a human epidermoid carcinoma, were grown in RPMI 1640 plus 5% heat inactivated fetal calf serum (GIBCO). THP-1 (ATCC TIB202) and U-937 cells (ATCC CRL1593.2), both human monocytic cell lines, were grown in RPMI 1640 plus 10% heat-inactivated fetal calf serum. RAW264.7 (ATCC TIB71) and J774A.1 (ATCC TIB67), both murine cell lines, were grown in DMEM plus 10% heat-inactivated fetal calf serum.

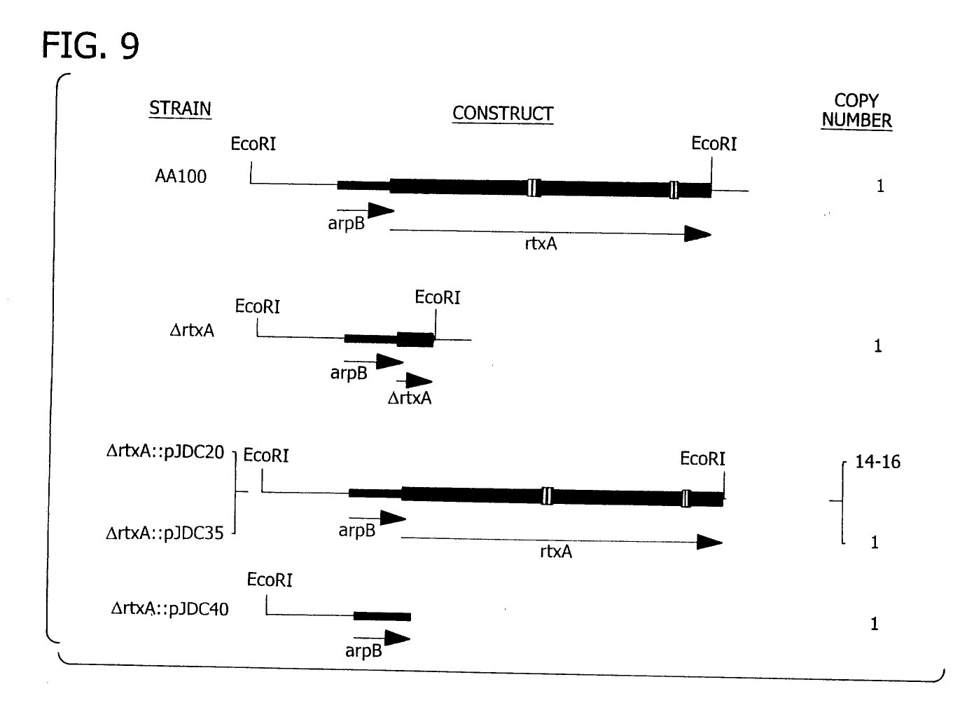

Molecular techniques and plasmid construction. The previous example 1 demonstrated that the rtxA gene affects entry into epithelial cells (Hep-2) and monocytes (THP-1). In the current experiment, the wild-type L. pneumophila strain AA100, ΔrtxA mutant and complementing strains were examined for other phenotypic characteristics that may help to determine whether rtxA plays a role in virulence. The structure of the in-frame deletion in rtxA and the constructs used for complementation are shown in FIG. 9. Both single-copy and multi-copy complementation constructs were used to control for enhanced expression of rtxA due to copy-number effects. To ensure physiologically normal RtxA levels for complementation we expressed rtxA from its endogenous promoter in the same position as it is found in the L. pneumophila chromosome. Since sequence analysis of the rtxA region suggests that the gene is the second in an operon of two genes, we utilized complementing constructs that contain the entire operon and putative promoter (pJDC20 and pJDC35). In order to determine whether complementation is solely due to the presence of the rtxA gene on this construct, an identical construct without rtxA was also used (pJDC40). The combination of these constructs allows definitive demonstration of the activity of the rtxA gene under conditions that are as close as possible to those that naturally occur in the L. pneumophila chromosome.

DNA manipulations were carried out essentially as described previously (49). See Sambrook, et al., Molecular cloning: a laboratory manual, 2 ed., Cold Spring Harbor Laboratory, Cold Spring Harbor, N.Y. (1989). The construction of the ΔrtxA mutant is described previously in example 1. The complementation plasmid pJDC20 (FIG. 1) carries the EcoRI fragment that contains only the enh1 locus with rtxA and putative promoter region. The L. pneumophila suicide plasmid pJDC35 (FIG. 9) was constructed by insertion of this same EcoRI fragment into the EcoRI site of pJDC15. The L. pneumophila suicide plasmid pJDC40 (FIG. 9) was constructed by digestion of pJDC35 with SwaI and EcoRV followed by self-ligation. Both pJDC35 and pJDC40 were propagated in ψec47 prior to transformation into L. pneumophila. Maintenance of these plasmids can only occur by integration into the L. pneumophila chromosome via homologous recombination. The presence of the appropriate integrated plasmid was confirmed by Southern and PCR analysis of chromosomal DNA from the resulting strains as described previously.

Adherence assays. Adherence assays were carried out by the ‘immediate assay□ method described previously. See Cirillo, et al., “Intracellular growth in Acanthamoeba castellanii affects monocyte entry mechanisms and enhances virulence of Legionella pneumophila,” Infect. Immun. 67:4427-4434 (1999). HEp-2 cells were seeded in 24 well tissue culture dishes (Falcon) at a concentration of 1.5×10⁵ cells/well and allowed to adhere overnight at 37° C. After adding the bacteria at an MOI of 10-100, the media was gently mixed, washed five times with phosphate buffered saline (PBS) to remove non-adherent bacteria and then lysed by incubation for 10 minutes in one ml of water followed by vigorous pipetting. Although multiple MOIs (from 10-100) were used in these experiments, all data shown are for an MOI of 10. Within this 10-fold range, the MOI did not significantly affect the data obtained. In the case of THP-1 cells, the assays were carried out in suspension. This requires that the cells be pelleted by centrifugation at 100×g for one minute before each change of solution. After lysis the number of cell associated bacteria was determined by plating for colony forming units (cfu) on BCYE. Adherence assays on formaldehyde fixed cells were carried out in the same manner except that the cells were fixed in 3.7% formaldehyde for 10 minutes, washed three times with PBS and suspended in RPMI prior to addition of the bacteria. For formaldehyde fixed cells the bacteria were co-incubated with the cells for 30 minutes with THP-1 cells and 90 minutes with Hep-2 cells. Adherence levels were determined by calculating the percentage of the inoculum that became cell associated over the course of the assay (i.e. % adherence=100×(cfu cell associated/cfu inoculum)). In order to correct for variation in levels of uptake between experiments, adherence is reported relative to AA100 (i.e. relative adherence=% adherence test strain/% adherence AA100).

Intracellular growth assays. The 48 hour growth assays were carried out in a similar manner to that described elsewhere. See Zuckman, et al., “Pore-forming activity is not sufficient for Legionella pneumophila phagosome trafficking and intracellular growth,” Molec. Microbiol. 32:990-1001 (1999). The THP-1 cells used for growth assays were seeded into 24 well tissue culture dishes at 1.5×10⁶ cells/well in RPMI plus 10% serum and activated with γ-interferon (IFN) and lipopolysaccharide (LPS; Difco, E. coli 0127:B8) as described previously. For 48 hour growth assays bacteria were added to the cells and incubated at 37° C. for 1 hour, washed multiple times with warm PBS and suspended in fresh medium for 48 hours before lysis with water. Detailed growth assays were carried out as described previously. In these assays the bacteria were incubated with the host cells for 5 minutes and then treated in the same manner as the 48 hour assays with lysis at various times after washing. Dilutions of the resulting lysates were plated on BCYE agar to determine cfu immediately after the washes and at each time point. Growth is reported as the mean number of cfu present in triplicate samples at various times divided by the number of cfu present immediately after washing (Mean CFU Tx/To).

Cytotoxicity and pore-formation assays. The standard lactate dehydrogenase (LDH) release cytotoxicity assay was used in these studies. See Behl et al., “Hydrogen peroxide mediates amyloid beta protein toxicity,” Cell 77(6):817-27 (1994). The procedure used was essentially as recommended by the manufacturer of the CytoTox96 Non-Radioactive Cytotoxicity Assay system (Promega). Serial dilutions were made of each bacterial strain in a final volume of 100 μl for each assay using 2×10⁴ THP-1 or 2.5×10³ Hep-2 cells. Appropriate numbers of cells for CytoTox96 assays were determined as suggested by the manufacturer (Promega). The cells were incubated with the bacteria for 4 hours at 37° C.+5% CO₂ Cytotoxicity readings were taken using an ELISA plate reader at 450 nm. Percent cytotoxicity was calculated as recommended by the manufacturer and corrected for small differences in the inocula used.

Formation of pores in host cells was assayed by ethidium bromide and acridine orange staining in exactly the same manner as described previously using THP-1, U-937, RAW264.7, J774A.1 and Hep-2 cells. See Kirby et al., “Evidence for pore forming ability by Legionella pneumophila,” Mol. Microbiol. 27:323-336 (1998); Zuckman et al., “Pore-forming activity is not sufficient for Legionella pneumophila phagosome trafficking and intracellular growth,” Molec. Microbiol. 32:990-1001 (1999). Stained coverslips were examined using a Nikon TE300 inverted microscope with FITC and TRITC filters. Dual images of multiple fields were captured using an Optronics CCD video camera and analyzed the same manner as described previously. Pore-formation is expressed as the percentage of acridine orange stained cells that also stain with ethidium bromide resulting from incorporation of this dye into chromosomal DNA due to increased permeability of the host cell. All cells are stained with acridine orange since, unlike ethidium bromide, acridine orange readily crosses membranes of eukaryotic cells.

Mouse infections and pathological examination. In order to examine the virulence of the different L. pneumophila strains in mice, we used methods described previously. See Bermudex et al., “An animal model of Mycobactrium avium complex disseminataed infection after colonization of the intestinal tract,” J.Infect.Dis. 165:75-79 (1992); Brieland et al., Replicative Legionella pneumophila lung infections in intratracheally inoculated A/J mice: A murine model of human Legionnaires' disease,” Am.J.Pathol. 145:1537-1546 (1994). A/J mice were infected by intratracheal inoculation with 106 bacteria. The mice were harvested 1, 4, 24 and 48 hours after infection and bacteria in the lungs quantitated as described previously. Data represent the mean and standard deviation of cfu/g of lung from 12 mice in each experimental group. All preparations were suspended in PBS prior to inoculation.

Histopathology was conducted essentially as described previously. See Moffat et al., “Effects of an isogenic Zn-metalloprotease-deficient mutant of Legionella pneumophila in guinea-pig model,” Molec. Microbiol. 12:693-705 (1994). Lungs were fixed by immersion in 10% neutral phosphate-buffered formalin, processed routinely, embedded in paraffin, cut at 5 μm and stained with hematoxylin and eosin. Detection of L. pneumophila in sections was through the use of Warthin-Starry silver stain. See Kerr et al., “Improved Warthin-Starry method of staining spirochetes in tissue section,” Am.J.Clin.Pathol. 8:63-67 (1938). Each section was assigned a number code to allow blinded examination by light microscopy.

Statistical Analyses. All in vitro experiments were carried out in triplicate and repeated 3 times. The experiments in vivo were carried out using 12 mice per experimental group. Significance of the results was analyzed using ANOVA. Values of p<0.05 were considered significant.

### Results

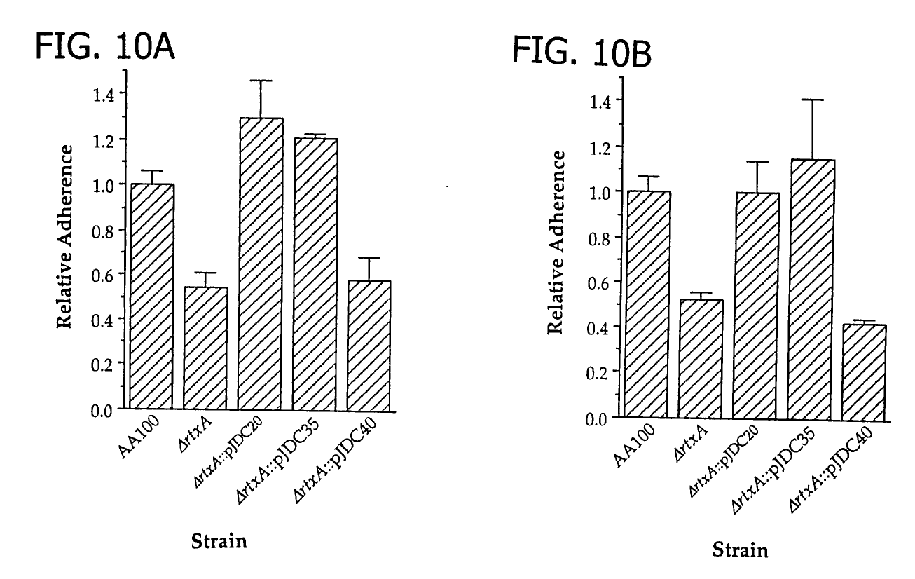

The rtxA gene affects adherence to monocytes and epithelial cells. The entry mechanism used by L. pneumophila may be the result of interaction of the host cell with the bacteria at the level of adherence, entry or a combination of these two events. The rtxA gene has been previously shown to play a role in entry into monocytes (THP-1) and epithelial (HEp-2) cells, but adherence and other phenotypic characteristics potentially related to virulence were not examined. Sodium and osmotic sensitivity of ΔrtxA was not significantly different from wild-type. In order to ascertain the role of adherence in rtxA-mediated entry, we examined the adherence of the rtxA mutant to epithelial and monocytic cells (FIG. 10). Adherence to both cell types was reduced by approximately 50% in the rtxA mutant as compared to wild-type L. pneumophila. In contrast, levels of adherence similar to wild-type were observed in the rtxA mutant carrying a complementing construct containing the complete rtxA gene. However, the rtxA mutation could not be complemented by the same construct containing the putative promoter region and complete arpB gene in the absence of rtxA. All strains were tested for sensitivity to assay conditions such as osmotic lysis, culture medium and serum. No significant differences were observed between ΔrtxA and wild-type. These data suggests that the rtxA gene is involved in adherence of L. pneumophila to epithelial and monocytic cells.

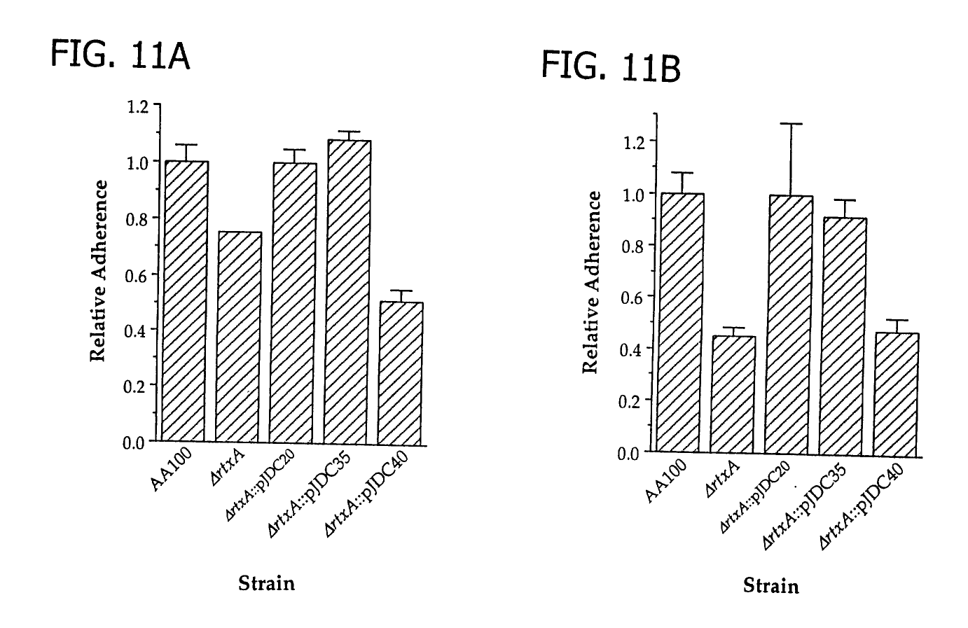

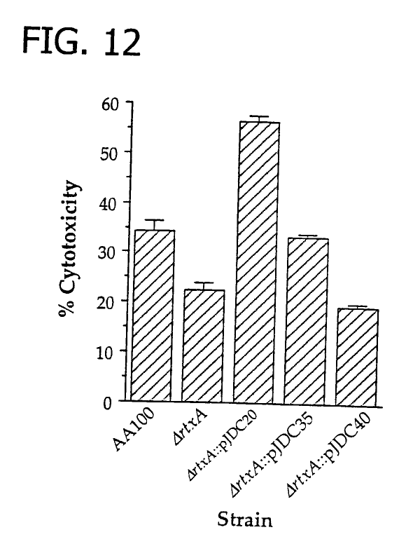

Although adherence assays are carried out quickly to prevent the possibility of intracellular killing by the host cell, we initially felt that it was possible that some portion of the difference in adherence observed is due to affects on survival after internalization. In order to test this possibility, we examined the adherence of the rtxA mutant to formaldehyde fixed epithelial and monocytic cells (FIG. 11) where internalization can not occur. Similar differences were observed between the rtxA mutant and wild-type L. pneumophila strains. Since we were concerned about the effects of formaldehyde fixation on membrane fluidity and other factors related to adherence, such as the structure of the host cell receptor, we examined the effects of fixation on the number of extracellular bacteria obtained in this assay as compared to the ‘immediate assay□. Both assay methods result in nearly all bacteria remaining extracellular (99.7-99.9%), where they are killed by gentamicin. Thus, both assay methods are sufficient to allow evaluation of the role of rtxA in adherence and killing subsequent to uptake does not contribute significantly to the data obtained. This information suggests that the preferred assay for adherence of L. pneumophila would be the immediate assay since it results in nearly all bacteria remaining extracellular and is unlikely to have aberrant affects on the host cell.

The rtxA gene is involved in cytotoxicity and pore-formation caused by L. pneumophila. One common characteristic of RTX proteins from other bacterial species is their involvement in pore-forming cytotoxicity for eukaryotic cells. See Welch et al., “Pore-forming cytolysin of gram-negative bacteria,” Molec. Microbiol. 5:521-528 (1991). Genes involved in pore-forming cytotoxicity have recently been associated with the ability of L. pneumophila to replicate intracellularly. See Vogel et al., “Conjugative transfer by the virulence system of Legionella pneumophila,” Science 279:(5352):873-6 (1998). Thus, the rtxA gene is a potential mediator of the cytotoxicity and/or pore-formation associated with L. pneumophila infection. When we examined the role of rtxA in cytotoxicity, we found that the ΔrtxA mutant displayed less cytotoxicity for monocytic cells than wild-type (FIG. 12). The level of cytotoxicity observed in wild-type L. pneumophila (˜35%) was less than that observed previously (˜65%) using a similar assay. These differences are likely due to differences in the cell lines (bone marrow-derived murine macrophages vs. THP1 human monocytes) and bacterial strains (LpO2 vs. AA100) used. No cytotoxicity was observed for the epithelial cell line, HEp-2, with wild-type or the rtxA mutant strain. This data suggests that the cytotoxicity of L. pneumophila is at least somewhat specific for monocytes. However, the fact that rtxA affects cytotoxicity is not necessarily directly related to the pore-formation previously observed during L. pneumophila infection of monocytes.

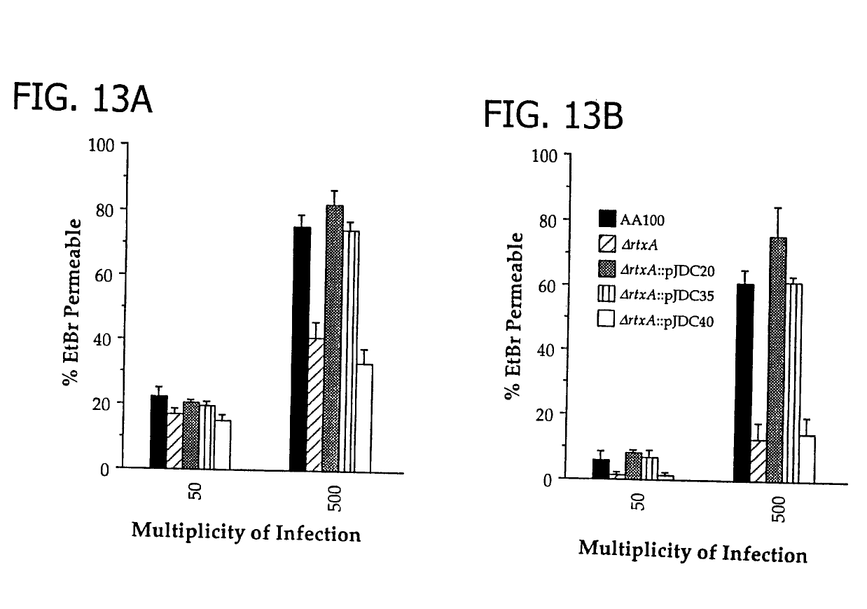

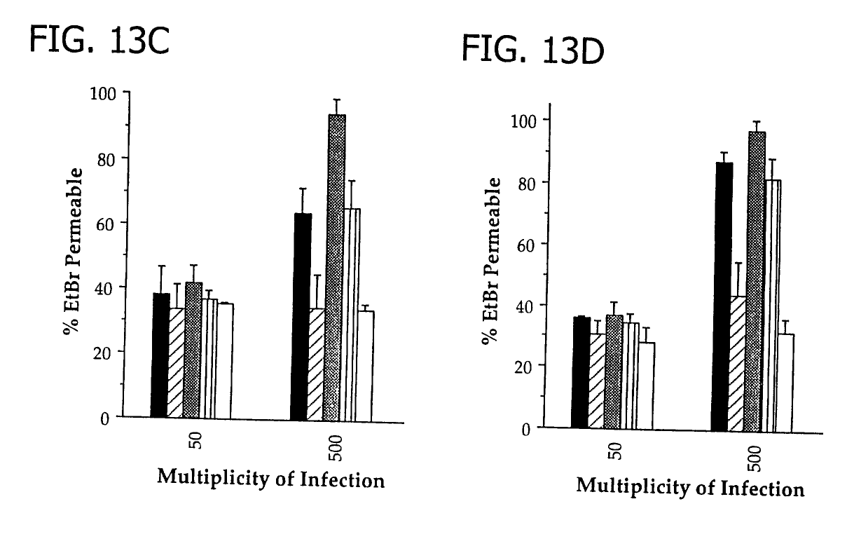

In order to determine the role of rtxA in pore-formation, we compared the pore-forming ability of wild-type L. pneumophila with that of the rtxA mutant and complemented clones in four different monocytic cell lines (FIG. 13). We utilized both human and murine cells for these assays to determine whether the pore-forming activity was species specific, as is sometimes observed with RTX proteins from other bacterial species. Our data indicate that rtxA is involved in a pore-forming activity that occurs in both murine and human monocytes. Although the level of pore-formation varies in different cell types, pore-formation is consistently reduced in the rtxA mutant as compared to wild-type and correlates with increased bacteria per cell ratios. The smallest difference is observed in RAW264.7 cells, a mouse macrophage cell line, though this difference is still significant (p=0.043). No pore-formation was observed in HEp-2 cells with wild-type L. pneumophila or the rtxA mutant (data not shown). These data indicate that the L. pneumophila rtxA gene is involved in a cytotoxic and pore-forming activity affecting both human and murine monocytic cells but not HEp-2 cells.

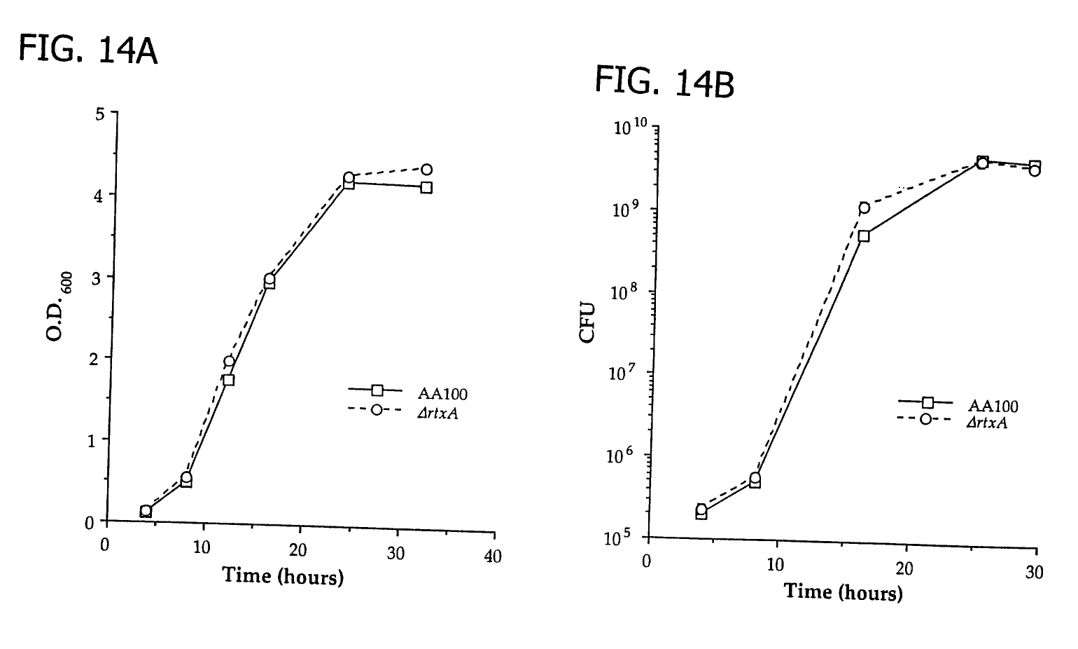

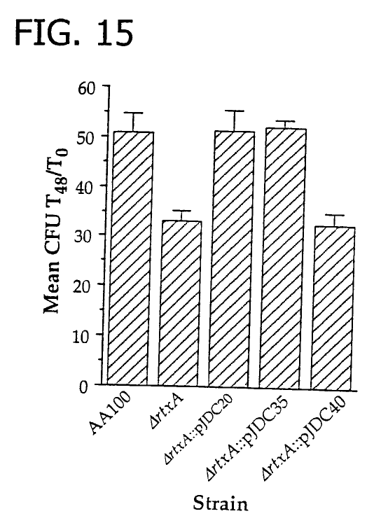

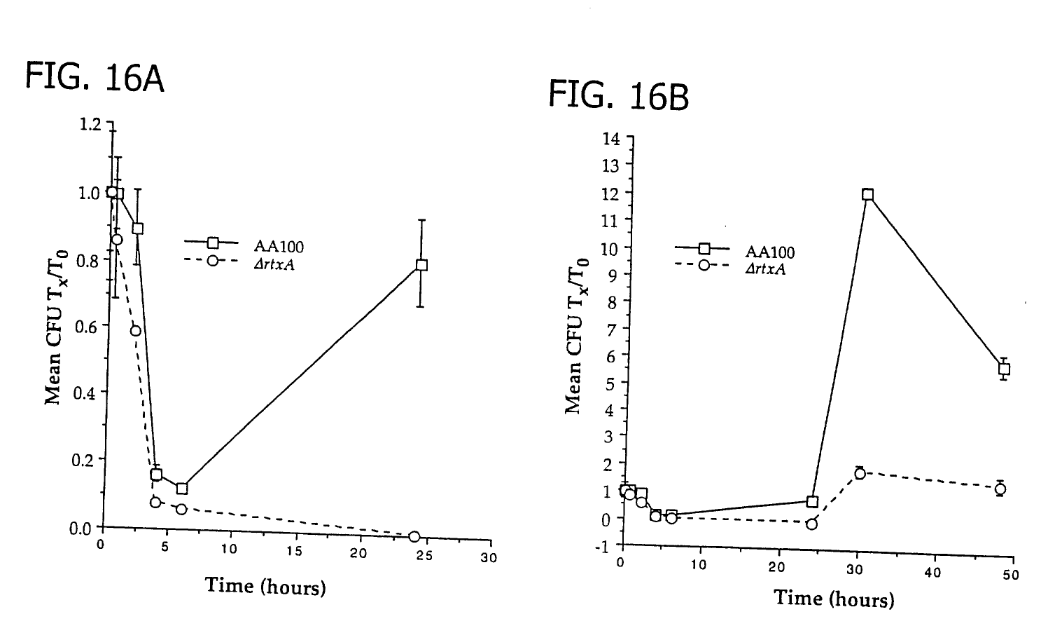

Optimal intracellular survival and replication require rtxA. Since pore-formation is thought to be involved in the intracellular survival of L. pneumophila, the activities that are associated with rtxA may also affect intracellular viability. In order to elucidate whether rtxA plays an important role early during intracellular infection we compared the ability of the ΔrtxA mutant to survive and replicate in monocytes. Although the replication of the ΔrtxA mutant is the same as wild-type in BYE broth (FIG. 14), growth in monocytes is significantly lower during the first 48 hours of growth (FIG. 15). In order to examine the intracellular viability of L. pneumophila at very early time points during intracellular growth we used a five minute co-incubation period with host cells. This procedure allowed detailed examination of the kinetics of intracellular growth from five minutes to 48 hours (FIG. 16). This data demonstrates that the ΔrtxA mutant appears to be killed more efficiently in monocytes during the first 2.5 hours after uptake. The apparent difference between the fold increase observed in FIG. 15 and FIG. 16 is due to the different time zero used (1 hour as opposed to 5 minutes) along with the rapid intracellular killing observed at early time points during intracellular growth. Taking these two factors into consideration, the data are consistent with AA100 increasing from approximately 0.1 to 6 (60 fold) and ΔrtxA increasing from approximately 0.05 to 2 (40 fold) over 48 hours. The early intracellular killing of the ΔrtxA mutant suggests that the effects of rtxA on entry affect intracellular viability. This early defect in intracellular survival leads to continuously lower intracellular replication, even at time points as late as 48 hours. Thus, it may not be possible to fully rescue organisms that do not communicate properly with the host cells early on in their intracellular life cycle.

The rtxA gene affects virulence. Since the differences between ΔrtxA and wild-type are relatively small in these in vitro assays, we wished to determine whether these small differences in phenotype significantly affect the ability of L. pneumophila to cause disease. In order to elucidate whether the phenotypic effects observed in vitro correlate with changes in virulence, we compared the ability of the wild-type, ΔrtxA mutant and the complemented strains to infect mice. The results are listed in Table 2 below.

TABLE 2
Table 2. Replication of L. pneumophila in lungs after intratracheal inoculation.

| Hours | | | | | |
|---|---|---|---|---|---|
| After | | | | | |
| Infec- | Strain (cfu/g of lung) | | | | |
| tion | AA100 | ΔrtxA | ΔrtxA::pJDC20 | ΔrtxA::pJDC35 | ΔrtxA::pJDC40 |
| 1 | 4.1 ± 0.3 × 10⁴ | 4.9 ± 0.3 × 10⁴ | 8.4 ± 0.2 × 10⁴ | 2.4 ± 0.3 × 10⁴ | 3.5 ± 0.2 × 10⁴ |
| 4 | 6.5 ± 0.3 × 10⁴ | 6.1 ± 0.2 × 10⁴ | 1.9 ± 0.2 × 10^{5b} | 5.9 ± 0.3 × 10⁴ | 3.1 ± 0.2 × 10⁴ |
| 24 | 8.3 ± 0.4 × 10⁴ | 5.8 ± 0.3 × 10^{3b} | 2.6 ± 0.4 × 10^{6b} | 6.4 ± 0.3 × 10⁴ | 8.4 ± 0.3 × 10^{3b} |
| 48 | 1.9 ± 0.2 × 10⁵ | 1.6 ± 0.2 × 10^{2b} | 1.2 ± 0.3 × 10^{7b} | 8.1 ± 0.3 × 10⁴ | 1.2 ± 0.4 × 10^{3b} |

^{a}An inoculum of 10⁶ bacteria was used for all experimental groups. Data represents the means ± standard deviations of duplicate platings from 12 mice.
^{b}Significantly different (P < 0.05) than wild-type L. pneumophila (AA100).

Although the initial number of bacteria found in the lung one hour after infection is similar for all strains, the colony forming units (cfu) for the ΔrtxA mutant decrease over time. By 24 hours after infection there is a one log difference between the ΔrtxA mutant and wild type; whereas, there was no significant difference between the single-copy complemented strain (ΔrtxA::pJDC35) and wild-type at any time point. This corresponds well with our observation that the ΔrtxA mutant displays reduced intracellular survival in monocytes. Interestingly, the cfu for ΔrtxA mutant containing the multi-copy plasmid pJDC20 increase more quickly than for wild-type, suggesting that the increased copy number of this region enhances the ability of L. pneumophila to survive and/or replicate in mouse lungs.

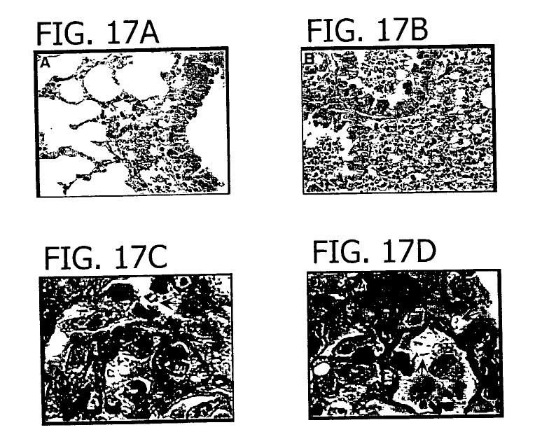

Throughout the course of these experiments the animals were monitored for signs of disease. At 1 hour after infection no mice displayed any adverse symptoms. However, by 48 hours all of the mice infected with wild-type (AA100) and ΔrtxA::pJDC20 and the majority of the mice infected with ΔrtxA::pJDC35 displayed malaise and ruffled fur; whereas, only 1 mouse infected with the ΔrtxA mutant showed malaise. Histopathologic examination of lungs from mice infected with these strains (FIG. 17) confirmed the disease state of the mice in each group and show characteristics similar to previous studies on L. pneumophila infections in mice. See Brieland et al., “Replicative Legionella pneumophila lung infections in intratracheally inoculated A/J mice: A muring model of human Legionnaires' disease,” Am.J.Pathol. 145:1537-1546 (1994). Lung tissue from mice infected with wild-type AA100, ΔrtxA::pJDC20 and ΔrtxA::pJDC35 displayed lesions consisting of lobular areas of parenchymal consolidation characterized by severe suppurative inflammation together with peribronchial and perivascular interstitial edema. Infiltration by mixed inflammatory cells, primarily polymorphonuclear neutrophils, was also observed. Large clusters of leukocytic exudate mixed with necrotic cellular debris and red blood cells are present in bronchiolar lumena and extended to the surrounding alveolar air spaces. However, the respiratory mucosa remain intact. Silver stain sections from mice infected with the wild-type strain display small clusters of intracellular rod-shaped organisms in mononuclear inflammatory cells present within affected alveolar lumena (FIG. 17). In contrast, lung tissues from the ΔrtxA mutant and ΔrtxA::pJDC40 are negative for lesions and bacteria are often extracellular and less abundant throughout the sections. These data indicate that the rtxA gene is a key virulence determinant and that the mechanism of entry used is likely to be critical to L. pneumophila pathogenesis.

Discussion

Monocytes utilize a number of relatively non-specific mechanisms to phagocytose particles including LPS-, surfactant-, Fc-, complement- and mannose-mediated mechanisms. In addition, pathogens can trigger specific mechanisms to enter monocytes. However, little is known about the affects of different entry mechanisms on subsequent intracellular viability of pathogens. We are interested in determining the affects of different entry mechanisms on the pathogenesis of L. pneumophila. Through the identification of the L. pneumophila genes involved in entry and characterization of their role in the establishment of a preferred intracellular niche and production of disease, we hope to improve our understanding of the importance of different entry mechanisms. This information is likely to lead to novel methods for the prevention of the disease process prior to invasion, the first step in pathogenesis, before an infection can become well established. The rtxA gene was initially identified because of its role in entry. However, in the current example we demonstrate that this gene also affects a number of other phenotypic characteristics potentially associated with pathogenesis including virulence in mice.

Although the rtxA gene affects adherence to epithelial cells, it is more critical for adherence to monocytes. This observation may provide some insight into potential host cell receptors used by the RtxA-mediated adherence mechanism. The β₂ integrin receptor has been shown to be a receptor for RTX proteins from other bacteria. See Ambagala et al., “The leukotoxin of Pasteurella haemolytica binds to β2 integrins on bovine leukocytes,” FEMS Microbiol Lett 179:161-167 (1999); Lally et al., “RTX toxins recognize a β2 integrin on the surface of human target cells,” J.Biol. Chem. 272:30463-30469 (1997). Thus, if RtxA binds to a similar receptor, our results may be explained by the fact that epithelial cells normally express much lower levels of β₂ integrins than monocytic cells. This model fits well with previous data demonstrating a role for complement receptors in adherence and entry. See Marra et al., “The HL-60 model for the interaction of human macrophages with the Legionnaires' disease bacterium,” J.Immunol. 144:2738-2744 (1990). The absence of observable pore-formation and cytotoxicity in HEp-2 cells may also be due to the potential involvement β₂ integrins in these events. The lack of rtxA cytotoxicity for epithelial cells could explain the histopathological observation of the maintenance of an intact respiratory mucosa in the presence of a severe inflammatory response. This pathologic observation is consistent with previous studies on L. pneumophila infections in guinea pigs. However, additional experiments are necessary to clearly demonstrate that the RtxA-mediated entry mechanism occurs via β₂ integrins. The ΔrtxA mutant isolated in the current studies should greatly facilitate further research into the role of host cell receptors and signaling pathways in L. pneumophila adherence and entry mechanisms.

It is possible that all of the other phenotypic characteristics that are associated with the ΔrtxA mutant are related to adherence and/or entry. Hypothetically, the mechanism of entry triggered by rtxA could result in signaling events that affect intracellular trafficking. These effects may be responsible for the ability of L. pneumophila to inhibit lysosomal fusion. Thus, the role of rtxA in intracellular survival may be explained through affects on trafficking. Examination of the intracellular trafficking of the ΔrtxA mutant after uptake into monocytes should allow us to obtain a better understanding of the role of the rtxA gene in pathogenesis. It has been shown that L. pneumophila replicates primarily within monocytes during disease. Despite the fact that the phenotypic affects of ΔrtxA in vitro were relatively small, approximately 2-fold, the affects in vivo were quite obvious, approximately 100-fold by 48 hours. Thus, subtle defects in the ability of L. pneumophila to enter, survive and replicate in monocytes in vitro might be expected to affect the ability to cause disease in humans. These data suggest that it is important to carefully examine potential virulence determinants in vitro and underscores the importance of virulence studies in animals where the selection for optimal growth of a pathogen may be more stringent.

The effects of rtxA on adherence may be directly responsible for the defect in the persistence of the ΔrtxA mutant in mouse lungs. However, it is equally possible that the rtxA gene has dual functions, adherence and pore-formation, both of which may be important for the pathogenesis of L. pneumophila. Proper intracellular trafficking, cytotoxicity and prevention of lysosomal fusion by L. pneumophila are thought to be due to a pore-forming activity involving a type IV secretion apparatus. Since RTX proteins are known to cause pore-formation in host cells it is possible that the rtxA gene product is responsible for this activity. Our observation of a pore-forming activity that requires the presence of the rtxA gene supports this hypothesis. However, it is unlikely that rtxA is solely responsible for the cytotoxicity associated with the dot/icm complex since an rtxA mutation only partially reduces cytotoxicity (˜37% reduction); whereas, dot/icm mutations more significantly reduce cytotoxicity (˜62% reduction). Furthermore, there are additional cytotoxic and haemolytic proteins that are known to be produced by L. pneumophila. It should be possible to construct a conditional mutant to modulate the rtxA phenotype in order to determine whether this gene has dual functions or its affects upon entry are sufficient to cause the other phenotypic affects observed. However, the rtxA gene is clearly involved in adherence to and entry into monocytes and is critical for the ability of L. pneumophila to survive and replicate in vivo.

What is claimed is:

1. A method of isolating and identifying bacterial genes that relate to virulence determinants, the method comprising:
(a) obtaining a pool of mutants of bacteria that over-express DNA of wild-type bacteria;
(b) selecting a mutant bacteria from step (a) that displays the enhanced virulence phenotype during interaction with a host cell relative to the wild-type bacteria;
(c) isolating the DNA that contains a sequence that is a factor in the enhanced virulence phenotype in the bacteria selected in step (b);
(d) constructing random mutations in the DNA isolated in step (c);
(e) transferring the DNA of step (d) into wild-type bacteria;
(f) selecting the bacteria of step (e) that no longer display the enhanced virulence phenotype in order to identify an active site of the DNA necessary to confer the enhanced virulence phenotype to the wild-type bacteria;
(g) constructing mutations in the active site of the DNA identified in step (f);
(h) transferring the DNA of step (g) into wild-type bacteria;
(i) comparing the resulting phenotype and DNA of the mutant bacteria from step (h) to the wild-type bacteria to identify the DNA responsible for the enhanced virulence phenotype.

2. A method of claim 1 wherein said bacterial genes are isolated and identified from Legionella pneumophila.

3. A method of claim 1 wherein said mutants of bacteria of step (a) are created using chemical mutagenesis.

4. A method of claim 1 wherein said mutants of bacteria of step (a) are created using ethylmethane sulfonate.

5. A method of claim 1 wherein said mutants of bacteria of step (a) are created using transposon mutagenesis.

6. A method of claim 1 wherein said mutants of bacteria of step (a) are created using random integrating controlled expression library comprising:
(a) isolating DNA from the wild-type bacteria;
(b) constructing a library from said DNA in a plasmid that does not replicate in the wild type bacteria, carries a promoter that functions in the wild-type bacteria located immediately upstream of the site for insertion of said DNA;
(c) transferring said library into the wild-type bacteria.

7. A method of claim 6 wherein said promoter is regulated.

8. A method of claim 6 wherein said promoter is not regulated.

9. A method of claim 1 wherein said mutants of bacteria of step (a) are created using a library constructed in a replicating plasmid.

10. A method of creating the replicating plasmid of claim 10 comprising:
(a) isolating the total DNA from the bacteria of interest;
(b) constructing a library from said isolated DNA in a plasmid that replicates in the wild-type bacteria;
(c) transferring said library into the wild-type bacteria.

11. A method of claim 10 wherein said plasmid carries a promoter that functions in the wild-type bacteria and is located immediately upstream of the site for insertion of said DNA.

12. A method of claim 10 wherein said plasmid does not carry a promoter that functions in the wild-type bacteria.

13. A method of claim 1 wherein the selecting of mutant bacteria of step (b) comprises:
(a) growing host cells in a culture medium;
(b) adding said mutated bacteria of step (a) of claim 1 to the culture medium with the host cells;
(c) allowing interaction of said host cells and said mutated bacteria;
(d) washing the host cells;
(e) incubating said culture medium with an antibiotic added to said culture medium;
(f) washing said host cells with phosphate buffered saline;
(g) lysing said host cells;
(h) determining the number of intracellular bacteria by plating for colony forming units;
(i) determining the entry levels of the mutated bacteria of step (a) of claim 1 by calculating the percentage of said intracellular bacteria from the total bacteria added to the cells.

14. A method of claim 1 wherein the selecting of mutant bacteria of step (b) comprises:
(a) growing host cells in a culture medium;
(b) adding said mutated bacteria of step (a) of claim 1 to the culture medium with the host cells;
(c) allowing interaction of said host cells and said mutated bacteria;
(d) washing the host cells;
(e) incubating said culture medium with an antibiotic added to said culture medium;
(f) washing said host cells;
(g) lysing said host cells;
(h) determining the number of bacteria that have adhered to the cell by plating for colony forming units;
(i) determining the adherence levels of the mutated bacteria of step (a) of claim 1 by calculating the percentage of bacteria adhered to the cells from the total of the bacteria added to the cells.

15. A method of claim 1 wherein the selecting the mutant bacteria of step (b) comprises:
(a) growing host cells in a culture medium;
(b) adding said mutated bacteria of step (a) of claim 1 to the culture medium with the host cells;
(c) allowing interaction of said host cells and said mutated bacteria;
(d) washing the host cells;
(e) incubating said culture medium with an antibiotic added to said culture medium;
(f) washing said host cells;
(g) lysing a portion of the total amount of said host cells at different time intervals;
(h) determining the number of bacteria that have adhered to the cell by plating for colony forming units;
(i) determining the growth rate of the mutated bacteria of step (a) of claim 1 by calculating the number of bacteria adhered to the cells at the different time intervals.

16. A method of claim 1 wherein the selecting of mutant bacteria of step (b) comprises:
(a) infecting animals with said mutated bacteria of step (a) of claim 1;
(b) harvesting tissue of the infected animals of step (a) at different time intervals after infection;
(c) determining the number of bacteria per gram of tissue by plating for colony forming units;
(d) determining the growth rate of the mutated bacteria of step (a) of claim 1 by calculating the number of said bacteria per gram of tissue taken at said time intervals.

17. A method of claim 1 wherein said enhanced virulence phenotype of the bacteria of step (a) is the result of the over-expression of the gene sought to be isolated and identified.

18. A method of claim 1 wherein said enhanced virulence phenotype of the bacteria of step (a) is the result of the reduced-expression of the gene sought to be isolated and identified.

19. A method of claim 1 wherein said host cell is a mammalian cell.

20. A method of claim 19 wherein said host cell is a human cell.

21. A method of claim 19 wherein said host cell is an said host cell is a epithelial cell.

22. A method of claim 19 wherein said host ell is a monocyte.

23. A method of claim 1 wherein the random mutations of step (d) are made using a mini-Tn10 transposon.
